(19) United States
(12) Patent Application Publication
(10) Pub. No.: US 2006/0142269 A1
(43) Pub. Date: Jun. 29, 2006

(54) New compounds

(76) Inventor: Graeme Dykes, Berchem (BE)

Correspondence Address: FISH & RICHARDSON PC, P.O. BOX 1022, MINNEAPOLIS, MN, 55440-1022, US

(21) Appl. No.: 11/296,928

(22) Filed: Dec. 8, 2005

Related U.S. Application Data
(60) Provisional application No. 60/666,261, filed on Mar. 28, 2005.

(30) Foreign Application Priority Data
Dec. 9, 2004 (SE) 0403006-0

(51) Int. Cl. A61K 31/551; A61K 31/496; C07D 405/02

(52) U.S. Cl. 514/218; 540/575; 544/373; 544/376; 514/254.11

(57) ABSTRACT

The present invention relates to compounds of the general Formula (I), 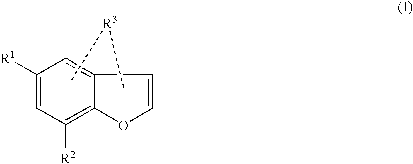
wherein R¹, R² and R³ are as defined in the description; to pharmaceutical compositions comprising these compounds; and to the use of the compounds for the prophylaxis and treatment of medical conditions relating to obesity, type II diabetes, and/or CNS disorders.

### RELATED APPLICATION INFORMATION

This application claims priority to U.S. provisional application Ser. No. 60/666,261, filed Mar. 28, 2005, and claims priority to Swedish application serial no. 0403006-0, filed Dec. 9, 2004, both of which are herein incorporated by reference.

### TECHNICAL FIELD

The present invention relates to novel compounds, to pharmaceutical compositions comprising the compounds, to processes for their preparation, as well as to the use of the compounds for the preparation of a medicament against 5-HT₆ receptor-related disorders.

### BACKGROUND OF THE INVENTION

Obesity is a condition characterized by an increase in body fat content resulting in excess body weight above accepted norms. Obesity is the most important nutritional disorder in the western world and represents a major health problem in all industrialized countries. This disorder leads to increased mortality due to increased incidences of diseases such as cardiovascular disease, digestive disease, respiratory disease, cancer and type 2 diabetes. Searching for compounds, which reduce body weight has been going on for many decades. One line of research has been activation of serotoninergic systems, either by direct activation of serotonin receptor subtypes or by inhibiting serotonin reuptake. The exact receptor subtype profile required is however not known.

Serotonin (5-hydroxytryptamine or 5-HT), a key transmitter of the peripheral and central nervous system, modulates a wide range of physiological and pathological functions, including anxiety, sleep regulation, aggression, feeding and depression. Multiple serotonin receptor subtypes have been identified and cloned. One of these, the 5-HT₆ receptor, was cloned by several groups in 1993 (Ruat, M. et al. (1993) Biochem. Biophys. Res. Commun.193: 268-276; Sebben, M. et al. (1994) NeuroReport 5: 2553-2557). This receptor is positively coupled to adenylyl cyclase and displays affinity for antidepressants such as clozapine. Recently, the effect of 5-HT₆ antagonist and 5-HT₆ antisense oligonucleotides to reduce food intake in rats has been reported (Bentley, J. C. et al. (1999) Br J Pharmacol. Suppl. 126, P66; Bentley, J. C. et al. (1997) J. Psychopharmacol. Suppl. A64, 255; Woolley M. L. et al. (2001) Neuropharmacology 41: 210-219). Compounds with enhanced affinity and selectivity for the 5-HT₆ receptor have been identified, e.g. in WO 00/34242 and by Isaac, M. et al. (2000) 6-Bicyclopiperazinyl-1-arylsulphonylindoles and 6-Bicyclopiperidinyl-1-arylsulphonylindoles derivatives as novel, potent and selective 5-HT₆ receptor antagonists. Bioorganic & Medicinal Chemistry Letters 10: 1719-1721 (2000), Bioorganic & Medicinal Chemistry Letters 13: 3355-3359 (2003), Expert Opinion Therapeutic Patents 12(4) 513-527 (2002).

### DISCLOSURE OF THE INVENTION

It has surprisingly been found that the compounds according to the present invention show affinity for the 5-HT₆ receptor as antagonists at nanomolar range. Compounds according to the present invention and their pharmaceutically acceptable salts have 5-HT₆ receptor antagonist, agonist and partial agonist activity, preferably antagonist activity, and are believed to be of potential use in the treatment or prophylaxis of obesity and type 2 diabetes, to achieve reduction of body weight and/or body weight gain, as well as in the treatment or prophylaxis of disorders of the central nervous system such as anxiety, depression, panic attacks, memory disorders, cognitive disorders, epilepsy, sleep disorders, migraine, anorexia, bulimia, binge eating disorders, obsessive compulsive disorders, psychoses, Alzheimer's disease, Parkinson's disease, Huntington's chorea and/or schizophrenia, panic attacks, Attention Deficit Hyperactive Disorder (ADHD), withdrawal from drug abuse (e.g. abuse of amphetamine, cocaine abuse and/or nicotine), neurodegenerative diseases characterized by impaired neuronal growth, and pain. The reduction of body weight and/or body weight gain (e.g. treating body-weight disorders) is achieved inter alia by reduction of food intake. As used herein, the term “body weight disorders” refers to the disorders caused by an imbalance between energy intake and energy expenditure, resulting in abnormal (e.g., excessive) body weight. Such body weight disorders include obesity.

The present invention provides a compound having the general Formula (I) 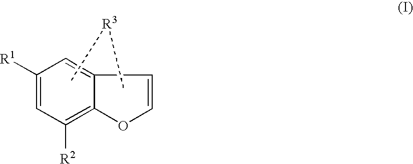
wherein
one of R¹ and R² is selected from Formula (II) or (III) 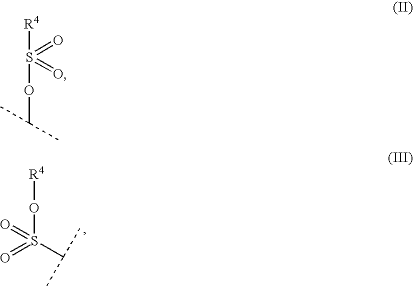
while the other one of R¹ and R² is selected from group of Formula (IV)-(XV): 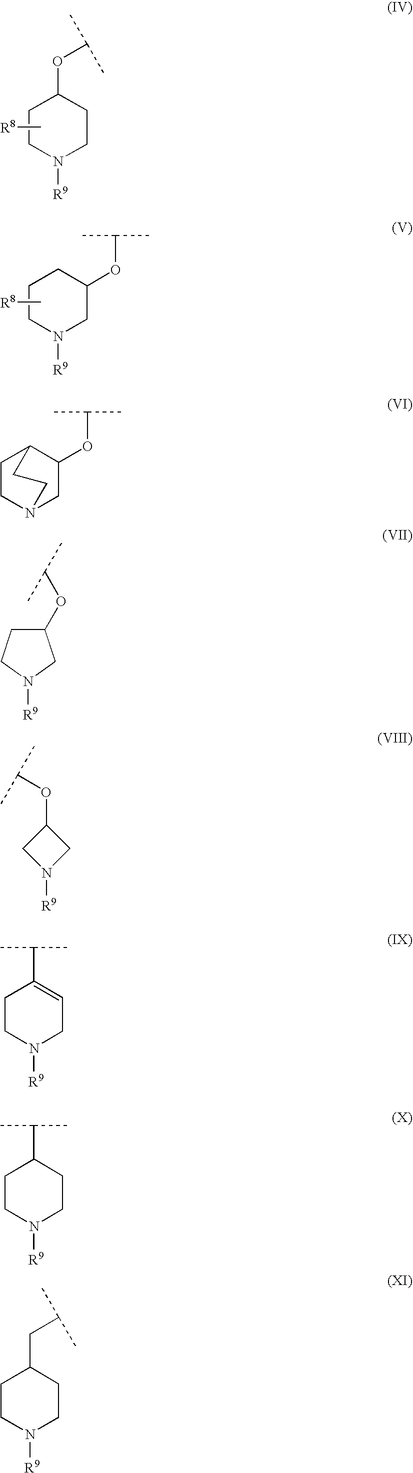 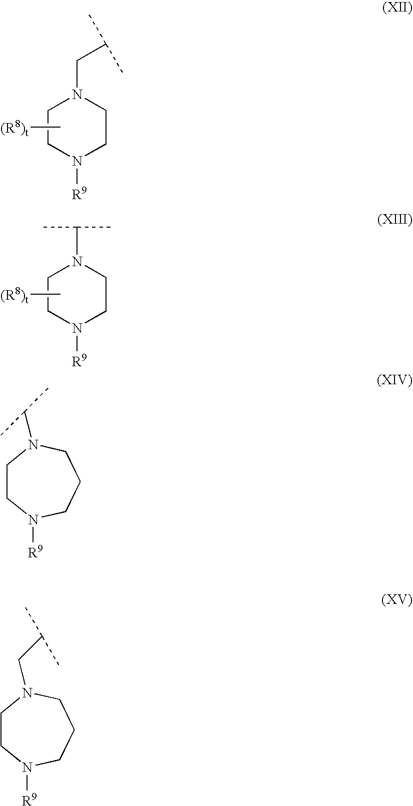
wherein:
t is 0, 1, or 2;
each R⁸ is independently
(a) hydrogen,
(b) methyl, or
(c) ethyl, and
when t=2, the R⁸ groups can be attached to the same or different carbon atom(s);
R⁹ is
(a) H,
(b) C₁₋₆ alkyl, or
(c) benzyl;
R³ is selected from
(a) hydrogen,
(b) C₁₋₄-alkyl,
(c) halogen, and
(d) C₁₋₄-alkoxy,
wherein the said R³ group is attached to a carbon atom in the 5-membered or the 6-membered ring;
R⁴ is selected from
(a) aryl,
(b) heteroaryl,
(c) heterocyclyl, provided that R¹ or R² is selected from a group of Formula (II),
(d) aryl-C₁₋₂-alkyl, provided that R¹ or R² is selected from a group of Formula (II), and
(e) cinnamyl, provided that R¹ or R² is selected from a group of Formula (II),
wherein any aryl and heteroaryl is optionally independently substituted in one or more positions with a substituent selected from
(a) halogen,
(b) C₁₋₆-alkyl,
(c) CF₃,
(d) C₁₋₆-alkoxy,
(e) C₂₋₆-alkenyl,
(f) phenyl,
(g) phenoxy,
(h) benzyloxy,
(i) benzoyl,
(j) —OCF₃,
(k —CN,
(l) hydroxy-C₁₋₄-alkyl,
(m) —CH₂—(CH₂)ₚF, wherein p is 0, 1, 2, or 3,
(n) —CHF₂,
(o) —NR⁵R⁵,
(p) —NO₂,
(q) —CONR⁵R⁵,
(r) —NHSO₂R⁷,
(s) —NR⁶COR⁷,
(t) —SO₂NR⁶R⁷,
(u) —C(═O)R⁷,
(v) —CO₂R⁶,
(z) —S(O)ₙR⁷, wherein n is 1 or 2,
(aa) C₁₋₆-alkylthio,
(ab) —SCF₃,
(ac) C₂₋₄-alkynyl, and
(ad) hydroxy;
R⁵ is each independently selected from
(a) H,
(b) C₁₋₆-alkyl, and
(c) C₃₋₇-cycloalkyl,
or two R⁵ groups together with the nitrogen to which they are attached form a heterocyclic ring (e.g. a heterocyclic ring selected from the group consisting of azetidine, pyrrolidine, piperidine, piperazine, morpholine, and thiomorpholine), and when the two R⁵ groups form a piperazine ring, the hydrogen bearing nitrogen of the piperazine ring may be optionally substituted with a group selected from
(a) C₁₋₄-alkyl,
(b) 2-cyanoethyl,
(c) hydroxy-C₂₋₄-alkyl,
(d) C₃₋₄-alkenyl,
(e) C₃₋₇-cycloalkyl,
(f) C₃₋₇-cycloalkyl-C₁₋₄-alkyl, and
(g) C₁₋₄-alkoxy-C₂₋₄-alkyl;
R⁶ is each independently selected from
(a) hydrogen, and
(b) C₁₋₄-alkyl;
R⁷ is each independently selected from
(a) C₁₋₆-alkyl
(b) aryl, and
(c) heteroaryl,
wherein any heteroaryl or aryl residue is optionally independently substituted with one or more substituents selected from
(a) halogen,
(b) C₁₋₄-alkyl,
(c) C₁₋₄-alkylthio,
(d) C₁₋₄-alkoxy,
(e) —CF₃, and
(f) —CN;
and pharmaceutically acceptable salts, hydrates, solvates, geometrical isomers, tautomers, optical isomers, and prodrug forms thereof.

The inventions also features compounds of formula (I) wherein,
R⁴ is selected from
(a) aryl,
(b) heteroaryl,
(c) heterocyclyl, provided that R¹ or R² is selected from a group of Formula (II),
(d) aryl-C₁₋₂-alkyl, provided that R¹ or R² is selected from a group of Formula (II), and
(e) cinnamyl,
wherein any aryl and heteroaryl is optionally independently substituted in one or more positions with a substituent selected from
(a) halogen,
(b) C₁₋₆-alkyl,
(c) CF₃,
(d) C₁₋₆-alkoxy,
(e) C₂₋₆-alkenyl,
(f) phenyl,
(g) phenoxy,
(h) benzyloxy,
(i) benzoyl,
(j) —OCF₃,
(k —CN,
(l) hydroxy-C₁₋₄-alkyl,
(m) —CH₂—(CH₂)ₚF, wherein p is 0, 1, 2, or 3,
(n) —CHF₂,
(o) —NR⁵R⁵,
(P) —NO₂,
(q) —CONR⁵R⁵,
(r) —NHSO₂R⁷,
(s) —NR⁶COR⁷,
(t) —SO₂NR⁶R⁷,
(u) —C(═O)R⁷,
(v) —CO₂R⁶,
(z) —S(O)ₙR⁷, wherein n is 1 or 2,
(aa) C₁₋₆-alkylthio,
(ab) 13 SCF₃, and
(ac) C₂₋₄-alkynyl.

Preferred are compounds of Formula (I) wherein R¹ is of Formula (III) 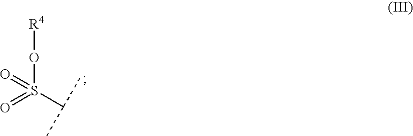
R² is selected from piperazinyl, homopiperazinyl, 2,6-dimethylpiperazinyl, 3,5-dimethylpiperazinyl, 2,5-dimethylpiperazinyl, 2-methylpiperazinyl, 3-methylpiperazinyl; 2,2-dimethylpiperazinyl, 3,3-dimethylpiperazinyl, piperidinyl, 1,2-unsaturated piperidinyl; 4-pyrrolidin-3-yloxy, 4-piperidinyloxy, 4-methylpiperazin-1-yl, homopiperazin-1-ylmethyl, 3-methylpiperazin-1-ylmethyl, and piperazin-1-ylmethyl;
R³ is hydrogen; and
R⁴ is selected from pyridinyl and phenyl,
wherein phenyl is optionally independently substituted in one or more positions with a substituent selected from:
(a) halogen,
(b) C₁₋₆-alkyl,
(c) CF₃,
(d) C₁₋₆-alkoxy, and
(q) CONR⁵R⁵.

Also within the invention are compounds of Formula (I) wherein
R¹ is of Formula (III) 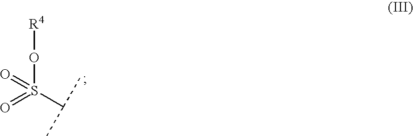
R² is selected from piperazinyl, homopiperazinyl, 2,6-dimethylpiperazinyl, 3,5-dimethylpiperazinyl, 2,5-dimethylpiperazinyl, 2-methylpiperazinyl, 3-methylpiperazinyl;
2,2-dimethylpiperazinyl, 3,3-dimethylpiperazinyl, piperidinyl, 1,2-unsaturated piperidinyl;
4-pyrrolidin-3-yloxy, 4-piperidinyloxy, and piperazinylmethyl;
R³ is hydrogen; and
R⁴ phenyl optionally independently substituted in one or more positions with a substituent selected from:
(a) halogen,
(b) C₁₋₆-alkyl,
(c) CF₃, and
(d) C₁₋₆-alkoxy.

Further preferred compounds of the general Formula (I) are compounds wherein
R¹ is selected from Formula (III) 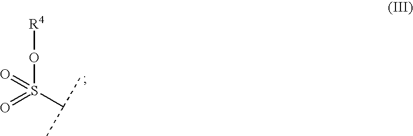
R² is selected from piperazinyl, homopiperazinyl, 3-methylpiperazinyl, 4-methylpiperazin-1-yl, homopiperazin-1-ylmethyl, 3-methylpiperazin-1-ylmethyl, and piperazin-1-ylmethyl;
R³ is hydrogen; and
R⁴ is selected from pyridinyl and phenyl,
wherein phenyl is optionally independently substituted in one or more positions with a substituent selected from:
(a) halogen selected from fluorine and chlorine
(b) C₁₋₄-alkyl,
(c) CF₃,
(d) C₁₋₄-alkoxy, and
(q) CONR⁵R⁵.

Yet further preferred compounds of the general Formula (I) are compounds wherein R¹ is selected from Formula (III) 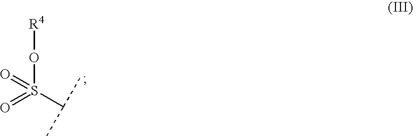
R² is selected from piperazinyl, homopiperazinyl, 3-methylpiperazinyl, 4-methylpiperazin-1-yl, homopiperazin-1-ylmethyl, 3-methylpiperazin-1-ylmethyl, and piperazin-1-ylmethyl;
R³ is hydrogen; and
R⁴ is selected from pyridinyl and phenyl,
wherein phenyl is optionally independently substituted in one or more positions with a
substituent selected from:
(a) chlorine
(b) methyl,
(c) CF₃,
(d) methoxy, and
(q) CONH₂.

Most preferred compounds of the generic Formula (I) are:
2-Methoxy-5-methylphenyl 7-piperazin-1-yl-1benzofuran-5-sulfonate,
2-Chlorophenyl-7-piperazin-1-yl-1benzofuran-5-sulfonate,
2-(Trifluoromethyl)-phenyl 7-piperazin-1-yl-1benzofuran-5-sulfonate,
Pyridin-3-yl 7-piperazin-1-yl-1-benzofuran-5-sulfonate,
2-Methoxy-5-methylphenyl 7-[(4-methylpiperazin-1-yl)methyl]-1-benzofuran-5-sulfonate,
2-Methoxy-5-methylphenyl 7-{[(3R)-3-methylpiperazin-1yl]methyl } -1benzofuran-5-sulfonate,
pyridin-3-yl 7-(4-methylpiperazin-1-yl)-1-benzofuran-5-sulfonate,
2,3-Dimethoxyphenyl 7-(4-methylpiperazin-1-yl)-1benzofuran-5-sulfonate,
2,3-Dimethoxyphenyl 7-[(3R)-3-methylpiperazin-1-yl]-1-benzofuran-5-sulfonate,
2,3-Dimethoxyphenyl 7-[(3S)-3-methylpiperazin-1-yl]-1-benzofuran-5-sulfonate,
3,5-Dimethoxyphenyl 7-(4-methylpiperazin-1-yl)-1benzofuran-5-sulfonate,
3,5-Dimethoxyphenyl 7-[(3R)-3-methylpiperazin-1-yl]-1-benzofuran-5-sulfonate,
3,5-Dimethoxyphenyl 7-[(3 S)-3-methylpiperazin-1yl]-1-benzofuran-5-sulfonate,
2-Methoxy-5-methylphenyl 7-{[(3S)-3-methylpiperazin-1-yl]methyl}-1-benzofuran-5-sulfonate,
2-(Aminocarbonyl)phenyl 7-{[(3S)-3-methylpiperazin-1-yl]methyl}-1-benzofuran-5-sulfonate,
2-(Aminocarbonyl)phenyl 7-{[(3R)-3-methylpiperazin-1-yl]methyl}-1-benzofuran-5-sulfonate,
2-Methoxy-5-methylphenyl 7-(piperazin-1-ylmethyl)-1-benzofuran-5-sulfonate,
2-methoxy-5-methylphenyl 7-(1,4-diazepan-1-ylmethyl)-1-benzofuran-5-sulfonate, and the pharmaceutically acceptable salts thereof.

Another object of the present invention is a process (A) for the preparation of a compound of Formula (I), comprising the following steps:
(a) Preparation of 7-substituted-2,3-dihydrobenzofuran-5-sulfonyl chloride from 2,3-dihydrobenzofuran-5-sulfonyl chloride and iodine monochloride;
(b) Oxidation of 7-substituted-2,3-dihydrobenzofuran-5-sulfonyl chloride with N-bromosuccinimide to provide 7-substituted benzofuran-5-sulfonyl chloride;
(c) Reacting a 7-substituted benzofuran-5-sulphonyl chloride intermediate, selected from 7-iodo-benzofuran-5-sulphonyl chloride, 7-bromo-benzofuran-5-sulphonyl chloride, 7-formyl-benzofuran-5-sulphonyl chloride or 7-hydroxy-benzofuran-5-sulphonyl chloride, with a hydroxy compound corresponding to R⁴OH, and
(d) Reacting the product from step c) with corresponding group selected from formula (IV)-(XV); and optionally thereafter forming a pharmaceutically acceptable salt of the compound of Formula (I).

Another object of the present invention is a process (A′) for the preparation of a compound of Formula (I), comprising the following steps:
(a) Preparation of 7-substituted-2,3-dihydrobenzofuran-5-sulfonyl chloride from 2,3-dihydrobenzofuran-5-sulfonyl chloride and iodine monochloride;
(b) Oxidation of 7-substituted-2,3-dihydrobenzofuran-5-sulfonyl chloride with N-bromosuccinimide to provide 7-substituted benzofuran-5-sulfonyl chloride;
(c) Esterification of 7-substituted benzofuran-5-sulphonyl chloride, with a hydroxy compound corresponding to R⁴OH, and
(d) Reaction of the product from step c) with corresponding group selected from formula (IV)-(XV); wherein said 7-substituted-benzofuran-5-sulphonyl chloride intermediates are selected from 7-iodo-benzofuran-5-sulphonyl chloride, 7-bromo-benzofuran-5-sulphonyl chloride, 7-formyl-benzofuran-5-sulphonyl chloride or 7-hydroxy-benzofuran-5-sulphonyl chloride.

Another object of the present invention is to provide a further process (B) for the preparation of a compound according to Formula (I), wherein R¹ is selected from Formula (III) and R² is selected from Formula (XIII) and (XIV), which process comprises the reaction of a 7-halo substituted benzofuran derivative of Formula (IIa), 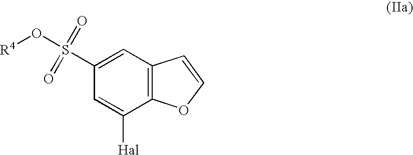
wherein R⁴ is as defined above, and Hal is selected from chloro, bromo and iodo, preferably iodo, with an appropriate secondary amine, or a protected derivative thereof, in the presence of a palladium catalyst together with an auxilliary ligand and a base, to give, optionally after deprotection, a compound of Formula (I), wherein R² is selected from Formula (XIII) and (XIV); and optionally thereafter forming a pharmaceutically acceptable salt of the compound of Formula (I).

Another object of the present invention is to provide a still further process (C) for the preparation of a compound according to Formula (I), wherein R¹ is selected from Formula (III) and R² is selected from Formula (XII) and (XV), which process comprises the following steps:
aa) reacting a 7-halo substituted benzofuran derivative of Formula (IIa), 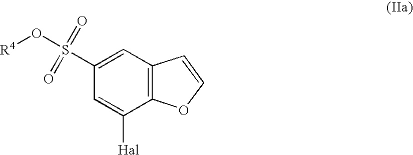
wherein R⁴ is as defined above, and Hal is selected from chloro, bromo and iodo, preferably iodo, with a metal cyanide salt, to give a compound of Formula (IIIa) 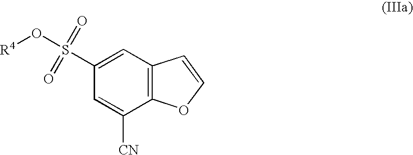
wherein R⁴ is as defined above;
bb) reacting the compound of Formula (IIIa) with a reducing agent, to give a compound of Formula (IVa) 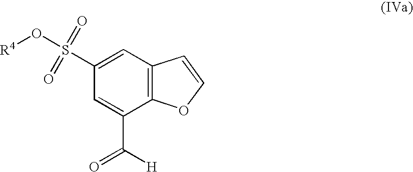
wherein R⁴ is as defined above;
cc) reacting the compound of Formula (IVa) with an appropriate secondary amine, or a protected derivative thereof, in the presence of a suitable reducing agent such as NaBH₄, NaBH₃CN or sodium triacetoxyborohydride [NaB(OAc)₃)H], to give, optionally after deprotection, a compound of Formula (I) wherein R² is selected from formula (XII) and (XV); and optionally thereafter forming a pharmaceutically acceptable salt of the compound of formula (I).

Another object of the present invention is to provide a yet further process (D) for the preparation of a compound according to formula (I), wherein R¹ is selected from formula (III) and R² is selected from formula (XII) and (XV), which process comprises the following steps:
aaa) reacting a 7-halo substituted benzofuran derivative of formula (IIa), 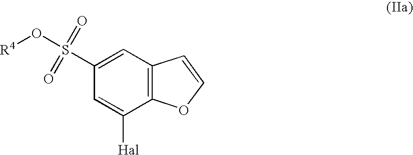
wherein R⁴ is as defined above, and Hal is selected from chloro, bromo and iodo, preferably iodo, with tributyl(vinyl)stannane in the presence of a palladium complex such as bis(triphenylphosphine)palladium(II) diacetate [Pd(PPh₃)₂OAc₂] as a catalyst, to give a compound of formula (Va) 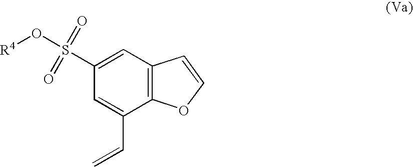
wherein R⁴ is as defined above;
bbb) reacting the compound of formula (Va) with osmium tetroxide (OSO₄) and sodium periodate, to produce the aldehyde derivative of formula (IVa) 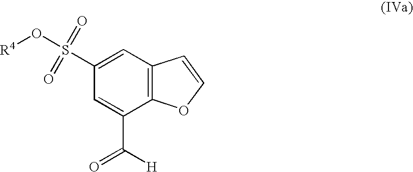
wherein R⁴ is as defined above;
ccc) reacting a compound of formula (IVa) according to Process C, step cc), described above; and optionally thereafter forming a pharmaceutically acceptable salt of the compound of formula (I).

Methods for carrying out the reactions described above are well known to those skilled in the art and/or are illustrated herein.

In Process A, step c), the reaction may be carried out in the presence of a base such as an alkali metal hydroxide such as, for example, an aqueous solution of sodium hydroxide, and a phase transfer catalyst such as benzyltrimethylammonium chloride or bromide in a solvent such as dichloromethane. See, for example: Synthesis 1979, 822-823 and J. Med. Chem. 2002, 45, 1086-1097.

In Process B the palladium-catalyzed amination may be conducted in the presence of a palladium catalyst such as tris(dibenzylideneacetone)dipalladium(0) [Pd₂dba₃] in conjunction with a ligand such as 9,9-dimethyl-4,6-bis(diphenylphosphino)xanthene (Xantphos) and a base such as sodium tert-butoxide in a solvent such as xylene, toluene or dioxane. See, for example: J. Org. Chem. 2004, 69, 8893-8902.

In Process C, step aa), the cyano derivative of formula (IIIa) may be prepared from the corresponding halo derivative, preferably iodo derivative, of formula (IIa) by reaction with a metal cyanide salt such as Zn(CN)₂ in the presence of a palladium-catalyst such as tetrakis(triphenylphosphine)palladium(0) [Pd(PPh₃)₄] in a solvent such as dimethylformamide (DMF). The reaction is typically performed under the influence of microwaves. See, for example: J. Org. Chem. 2000, 65, 7984-7989.

In Process C, step bb), the reduction of the nitrile group into an aldehyde function may be performed by aqueous formic acid in the presence of platinum(IV) oxide (PtO₂). See, for example: Tetrahedron Lett. 2002, 43, 1395-1396. Additionally, the reaction may optionally be carried out in the presence of a solvent such as tetrahydrofuran (THF).

In Process C, step cc), the reaction may be performed using standard methods for reductive amination. The reaction is typically performed in the presence of acetic acid in a solvent such as THF. See, for example: J. Org. Chem. 1996, 61, 3849-3862. Additionally, the reaction may optionally be conducted under the influence of microwaves.

In Process D, step aaa), the palladium-catalyzed cross-coupling reaction (Stille coupling) may be conducted in a solvent such as toluene or acetonitrile. The reaction may optionally be conducted under the influence of microwaves.

In Process D, step bbb), the oxidative cleavage of the alkene into an aldehyde function may be performed by conditions described in Organic Lett. 2004, 6, 3217-3219. The alkene is treated with osmium tetroxide/sodium periodate in a mixture of polar solvents such as dioxane and water in the presence of a base such as 2,6-lutidine.

In case the reacting amine in Process B, Process C, step cc), or Process D, step ccc), does possess additional primary or secondary amino nitrogens, a suitable protecting group such as tert-butoxycarbonyl (t-BOC) may be introduced prior to reaction in order to prevent undesired reactions at such primary or secondary amino nitrogens. An exemplary N-protected amine having more than one reactive nitrogen atom is N-tert-butoxycarbonylpiperazine. The said protecting group may be cleaved off when it is no longer needed to provide the compound according to Formula (I). The reaction conditions of removing the said protecting group depend upon the choice and the characteristics of this group. Thus e.g. tert-butoxycarbonyl may be removed by treatment with a suitable acid. Protecting group methodologies (protection and deprotection) are known in the art and are described in, for example, T. W. Greene and P. G. M. Wuts, Protective Groups in Organic Synthesis, 3^{rd} Ed., John Wiley & Sons (1999).

An obtained compound of Formula (I) may be converted to another compound of Formula (I) by methods well known in the art.

Another object of the present invention is a compound as mentioned above for use in therapy, especially for use in the treatment or prophylaxis of a 5-HT₆ receptor-related disorder, to achieve reduction of body weight and/or body weight gain.

Another object of the present invention is a pharmaceutical formulation comprising a compound as mentioned above as active ingredient, in combination with a pharmaceutically acceptable diluent or carrier, especially for use in the treatment or prophylaxis of a 5-HT₆ receptor-related disorder, to achieve reduction of body weight and/or body weight gain.

Another object of the present invention is a method for treating a human or animal subject suffering from a 5-HT₆ receptor-related disorder, to achieve reduction of body weight and/or body weight gain. The method can include administering to a subject (e.g., a human or an animal, dog, cat, horse, cow) in need thereof an effective amount of one or more compounds of any of the formulae herein, their salts, or compositions containing the compounds or salts.

The methods delineated herein can also include the step of identifying that the subject is in need of treatment of the 5-HT₆ receptor-related disorder, to achieve reduction of body weight and/or body weight gain. Identifying a subject in need of such treatment can be in the judgment of a subject or a health care professional and can be subjective (e.g., opinion) or objective (e.g., measurable by a test or diagnostic method).

Another object of the present invention is a method for the treatment or prophylaxis of a 5-HT₆ receptor-related disorder, to achieve reduction of body weight and/or body weight gain, which comprises administering to a subject in need of such treatment an effective amount of a compound as mentioned above.

Another object of the present invention is a method for modulating 5-HT₆ receptor activity, which comprises administering to a subject in need of such treatment an effective amount of a compound as mentioned above.

Another object of the present invention is the use of a compound as mentioned above for the manufacture of a medicament for use in the prophylaxis or treatment of a 5-HT₆ receptor-related disorder, to achieve reduction of body weight and/or body weight gain.

The compounds as mentioned above may be agonists, partial agonists or antagonists for the 5-HT₆ receptor. Preferably, the compounds act as partial agonists or antagonists for the 5-HT₆ receptor. More preferably the compounds act as antagonists for the 5-HT₆ receptor.

Examples of 5-HT₆ receptor-related disorders are obesity; type II diabetes; disorders of the central nervous system such as anxiety, depression, panic attacks, memory disorders, cognitive disorders, epilepsy, sleep disorders, migraine, anorexia, bulimia, binge eating disorders, obsessive compulsive disorders, psychoses, Alzheimer's disease, Parkinson's disease, Huntington's chorea, schizophrenia, attention deficit hyperactive disorder (ADHD), withdrawal from drug abuse (e.g. abuse of amphetamine, cocaine abuse and/or nicotine), neurodegenerative diseases characterized by impaired neuronal growth, and pain. The compounds and compositions are useful for treating diseases, to achieve reduction of body weight and/or body weight gain. The diseases include obesity; type II diabetes; disorders of the central nervous system such as anxiety, depression, panic attacks, memory disorders, cognitive disorders, epilepsy, sleep disorders, migraine, anorexia, bulimia, binge eating disorders, obsessive compulsive disorders, psychoses, Alzheimer's disease, Parkinson's disease, Huntington's chorea, schizophrenia, attention deficit hyperactive disorder (ADHD), withdrawal from drug abuse (e.g. abuse of amphetamine, cocaine abuse and/or nicotine), neurodegenerative diseases characterized by impaired neuronal growth, and pain. In one aspect, the invention relates to a method for treating or preventing an aforementioned disease comprising administering to a subject in need of such treatment an effective amount or composition delineated herein.

Another object of the present invention is a cosmetic composition comprising a compound as mentioned above as active ingredient, in combination with a cosmetically acceptable diluent or carrier, especially for use in the prophylaxis or treatment of a 5-HT₆ receptor-related disorder, to achieve reduction of body weight and/or body weight gain.

### DEFINITIONS

The following definitions shall apply throughout the specification and the appended claims.

Unless otherwise stated or indicated, the term “C₁₋₆-alkyl” denotes a straight or branched alkyl group having from 1 to 6 carbon atoms. Examples of said C₁₋₆-alkyl include methyl, ethyl, n-propyl, iso-propyl, n-butyl, iso-butyl, sec-butyl, t-butyl and straight- and branched-chain pentyl and hexyl. For parts of the range “C₁₋₆-alkyl” all subgroups thereof are contemplated such as C₁₋₅-alkyl, C₁₋₄-alkyl, C₁₋₃-alkyl, C₁₋₂-alkyl, C₂₋₆-alkyl, C₂₋₅-alkyl, C₂₋₄-alkyl, C₂₋₃-alkyl, C₃₋₆-alkyl, C₄₋₅-alkyl, etc. Likewise, “aryl-C₁₋₆-alkyl” means a C₁₋₆alkyl group substituted by one or more aryl groups.

Unless otherwise stated or indicated, the term “hydroxy-C₁₋₄-alkyl” denotes a straight or branched alkyl group that has a hydrogen atom thereof replaced with OH. Examples of said hydroxy-C₁₋₄-alkyl include hydroxymethyl, 2-hydroxyethyl, 2-hydroxypropyl and 2-hydroxy-2-methylpropyl.

Unless otherwise stated or indicated, the term “C₁₋₆-alkoxy” denotes a straight or branched alkoxy group having from 1 to 6 carbon atoms. Examples of said C₁₋₆-alkoxy include methoxy, ethoxy, n-propoxy, iso-propoxy, n-butoxy, iso-butoxy, sec-butoxy, t-butoxy and straight- and branched-chain pentoxy and hexoxy. For parts of the range “C₁₋₆-alkoxy” all subgroups thereof are contemplated such as C₁₋₅-alkoxy, C₁₋₄-alkoxy, C₁₋₃-alkoxy, C₁₋₂-alkoxy, C₂₋₆-alkoxy, C₂₋₅-alkoxy, C₂₋₄-alkoxy, C₂₋₃-alkoxy, C₃₋₆-alkoxy, C₄₋₅-alkoxy, etc.

Unless otherwise stated or indicated, the term “C₁₋₄-alkoxy-C₂₋₄-alkyl” denotes a straight or branched alkoxy group having from 1 to 4 carbon atoms connected to an alkyl group having from 1 to 4 carbon atoms. Examples of said C₁₋₄-alkoxy-C₂₋₄-alkyl include methoxymethyl, ethoxymethyl, iso-propoxymethyl, n-butoxymethyl, and t-butoxymethyl. For parts of the range “C₁₋₄-alkoxy-C₂₋₄-alkyl” all subgroups thereof are contemplated such as C₁₋₃-alkoxy-C₂₋₄-alkyl, C₁₋₄-alkoxy-C₂₋₃-alkyl, C₁₋₂-alkoxy-C₂₋₃-alkyl, C₂₋₄-alkoxy-C₂₋₄-alkyl, C₂₋₃-alkoxy-C₂₋₄-alkyl, C₂₋₄-alkoxy-C₂₋₃-alkyl, etc.

Unless otherwise stated or indicated, the term “C₂₋₆-alkenyl” denotes a straight or branched alkenyl group having from 2 to 6 carbon atoms. Examples of said C₂₋₆-alkenyl include vinyl, allyl, 2,3-dimethylallyl, 1-butenyl, 1-pentenyl, and 1-hexenyl. For parts of the range “C₂₋₆-alkenyl” all subgroups thereof are contemplated such as C₂₋₅-alkenyl, C₂₋₄-alkenyl, C₂₋₃-alkenyl, C₃₋₆-alkenyl, C₄₋₅-alkenyl, etc. Likewise, “aryl-C₂₋₆-alkenyl” means a C₂₋₆-alkenyl group substituted by one or more aryl groups. Examples of said aryl-C₂₋₆-alkenyl include styryl and cinnamyl.

Unless otherwise stated or indicated, the term “C₂₋₄-alkynyl” denotes a straight or branched alkynyl group having from 2 to 4 carbon atoms. Examples of said C₂₋₄-alkynyl include ethynyl, 1-propynyl, 2-propynyl, 1-butynyl, and 2-butynyl.

Unless otherwise stated or indicated, the term “C₃₋₇-cycloalkyl” denotes a cyclic alkyl group having a ring size from 3 to 7 carbon atoms. Examples of said cycloalkyl include cyclopropyl, cyclobutyl, cyclopentyl, cyclohexyl, methylcyclohexyl, and cycloheptyl. For parts of the range “C₃₋₇-cycloalkyl” all subgroups thereof are contemplated such as C₃₋₆-cycloalkyl, C₃₋₅-cycloalkyl, C₃₋₄-cycloalkyl, C₄₋₇-cycloalkyl, C₄₋₆-cycloalkyl, C₄₋₅-cycloalkyl, C₅₋₇-cycloalkyl, C₆₋₇-cycloalkyl, etc.

Unless otherwise stated or indicated, the term “aryl” refers to a hydrocarbon ring system of one, two or three rings, having at least one aromatic ring, and having from 6 to 14 ring carbon atoms. Examples of aryl groups include: phenyl, pentalenyl, indenyl, indanyl, 1,2,3,4-tetrahydronaphthyl, 1-naphthyl, 2-naphthyl, fluorenyl, anthryl, phenanthryl and pyrenyl. An aryl group can be linked to the remainder of the molecule through any available carbon atom in the aryl group whether present in an aromatic ring or a partially saturated ring.

The aryl rings may be optionally substituted. Likewise, aryloxy refers to an aryl group bonded to an oxygen atom.

The term “heteroaryl” refers to a mono- or bicyclic aromatic ring system, only one ring need be aromatic, and the said heteroaryl moiety can be linked to the remainder of the molecule via a carbon or nitrogen atom in any ring, and having from 5 to 10 ring atoms (mono- or bicyclic), in which one or more of the ring atoms are other than carbon, such as nitrogen, sulphur, oxygen and selenium. Examples of such heteroaryl rings include furyl, pyrrolyl, thienyl, oxazolyl, isoxazolyl, imidazolyl, thiazolyl, isothiazolyl, pyridinyl, pyrimidinyl, pyrazinyl, chromanyl, quinazolinyl, indolyl, isoindolyl, indolinyl, isoindolinyl, indazolyl, pyrazolyl, pyridazinyl, quinolinyl, isoquinolinyl, benzofuranyl, dihydrobenzofuranyl, benzodioxolyl, benzodioxinyl, benzothienyl, benzimidazolyl, benzothiazolyl, benzothiadiazolyl, and benzotriazolyl groups. If a bicyclic heteroaryl ring is substituted, it may be substituted in any ring.

Unless otherwise stated or indicated, the term “heterocyclic” refers to a non-aromatic (i.e., partially or fully saturated) mono- or bicyclic ring system having 4 to 10 ring atoms with at least one heteroatom such as O, N, or S, and the remaining ring atoms are carbon. Examples of heterocyclic groups include piperidyl, tetrahydropyranyl, tetrahydrofuranyl, azepinyl, azetidinyl, pyrrolidinyl, morpholinyl, imidazolinyl, thiomorpholinyl, pyranyl, dioxanyl, and piperazinyl groups. When present in heterocyclic groups, the sulfur atom may be in an oxidized form (i.e., S═O or O═S═O).

Unless otherwise stated or indicated, the term “halogen” shall mean fluorine, chlorine, bromine or iodine.

The term —S(O)ₙR⁷, wherein n is 1 or 2 has the meaning as illustrated by

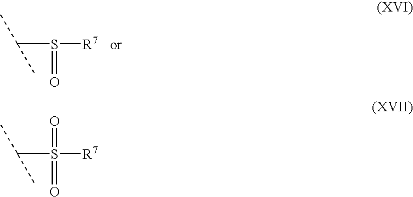

Formula (XVI or XVII): (XVI) (XVII)

“Optional” or “optionally” means that the subsequently described event or circumstance may but need not occur, and that the description includes instances where the event or circumstance occurs and instances in which it does not.

“Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect on the treated subject. The therapeutic effect may be objective (i.e., measurable by some test or marker) or subjective (i.e., subject gives an indication of or feels an effect).

The term “prodrug forms” means a pharmacologically acceptable derivative, such as an ester or an amide, which derivative is biotransformed in the body to form the active drug. Reference is made to Goodman and Gilman's, The Pharmacological basis of Therapeutics, 8^{th} ed., Mc-Graw-Hill, Int. Ed. 1992, “Biotransformation of Drugs”, p. 13-15; and “The Organic Chemistry of Drug Design and Drug Action” by Richard B. Silverman. Chapter 8, p 352. (Academic Press, Inc. 1992. ISBN 0-12-643730-0).

The following abbreviations have been used:

CV means Coefficient of Variation,

DMSO means dimethyl sulphoxide,

EDTA means ethylenediamine tetraacetic acid,

EGTA means ethylenebis(oxyethylenenitrilo)tetraacetic acid,

HEPES means 4-(2-hydroxyethyl)-1-piperazineethanesulfonic acid,

HPLC means high performance liquid chromatography,

LSD means lysergic acid, diethylamide,

MeCN means acetonitrile,

SPA means Scintillation Proximity Assay, and

THF means tetrahydrofuran,

ABS in Table 1 means absolute configuration,

MeOH means methanol,

p-ether means petroleum ether (40-60° C.),

R_{T} means retention time,

rt or r.t means room temperature,

t-BOC means t-butoxycarbonyl,

DCM means dichloromethane, and

TFA means trifluoroacetic acid.

All isomeric forms possible (pure enantiomers, diastereomers, tautomers, racemic mixtures and unequal mixtures of two enantiomers) for the compounds delineated are within the scope of the invention. Such compounds can also occur as cis- or trans-, E- or Z-double bond isomer forms. All isomeric forms are contemplated.

The compounds of the Formula (I) may be used as such or, where appropriate, as pharmacologically acceptable salts (acid or base addition salts) thereof. The pharmacologically acceptable addition salts mentioned above are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

For clinical use, the compounds of the invention are formulated into pharmaceutical formulations for oral, rectal, parenteral or other mode of administration. Pharmaceutical formulations are usually prepared by mixing the active substance, or a pharmaceutically acceptable salt thereof, with conventional pharmaceutical excipients. Examples of excipients are water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner.

In a further aspect the invention relates to methods of making compounds of any of the formulae herein comprising reacting any one or more of the compounds of the formulae delineated herein, including any processes delineated herein. The compounds of the Formula (I) above may be prepared by, or in analogy with, conventional methods.

The processes described above may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The compounds of Formula (I) may possess one or more chiral carbon atoms, and they may therefore be obtained in the form of optical isomers, e.g. as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

The chemicals used in the synthetic routes delineated herein may include, for example, solvents, reagents, catalysts, and protecting group and deprotecting group reagents. The methods described above may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations useful in synthesizing applicable compounds are known in the art and include, for example, those described in R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The necessary starting materials for preparing the compounds of Formula (I) are either known or may be prepared in analogy with the preparation of known compounds. The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

The invention will now be further illustrated by the following non-limiting Examples.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

TABLE 1

| Example | CHEMICAL NAME | R⁴ | R⁵ |
|---|---|---|---|
| 1 | 2-methoxy-5-methylphenyl 7- piperazin-1-yl-1-benzofuran- 5-sulfonate, trifluoroacetate | 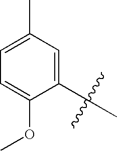 | 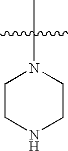 |
| 2 | 2-chlorophenyl 7-piperazin-1- yl-1-benzofuran-5-sulfonate, trifluoroacetate | 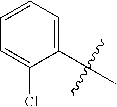 | 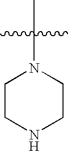 |
| 3 | 2-(trifluoromethyl)phenyl 7- piperazin-1-yl-1-benzofuran- 5-sulfonate, trifluoroacetate | 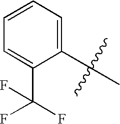 | 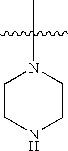 |
| 4 | pyridin-3-yl 7-piperazin-1-yl- 1-benzofuran-5-sulfonate, dihydrochloride | 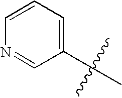 | 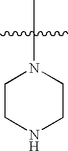 |
| 5 | 2-methoxy-5-methylphenyl 7- [(4-methylpiperazin-1- yl)methyl]-1-benzofuran-5- sulfonate, bis(trifluoroacetate) | 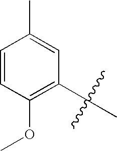 | 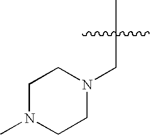 |
| 6 | 2-methoxy-5-methylphenyl 7- {[(3R)-3-methylpiperazin-1- yl]methyl}-1-benzofuran-5- sulfonate, bis(trifluoroacetate) | 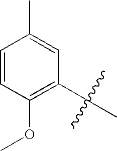 | 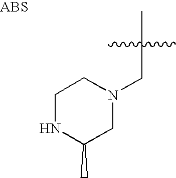 |
| 7 | pyridin-3-yl 7-(4- methylpiperazin-1-yl)-1- benzofuran-5-sulfonate, trifluoroacetate | 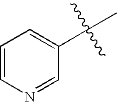 | 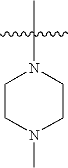 |
| 8 | 2,3-dimethoxyphenyl 7-(4- methylpiperazin-1-yl)-1- benzofuran-5-sulfonate, trifluoroacetate | 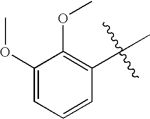 | 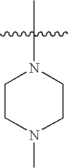 |
| 9 | 2,3-dimethoxyphenyl 7-[(3R)- 3-methylpiperazin-1-yl]-1- benzofuran-5-sulfonate, trifluoroacetate | 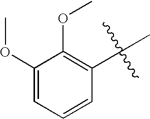 | 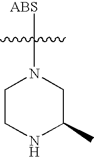 |
| 10 | 2,3-dimethoxyphenyl 7-[(3S)- 3-methylpiperazin-1-yl]-1- benzofuran-5-sulfonate, trifluoroacetate | 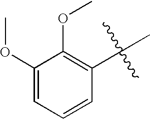 | 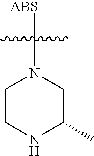 |
| 11 | 3,5-dimethoxyphenyl 7-(4- methylpiperazin-1-yl)-1- benzofuran-5-sulfonate, trifluoroacetate | 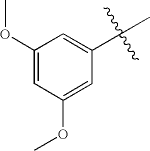 | 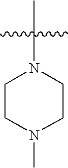 |
| 12 | 3,5-dimethoxyphenyl 7-[(3R)- 3-methylpiperazin-1-yl]-1- benzofuran-5-sulfonate, trifluoroacetate | 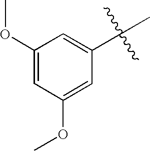 | 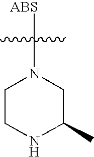 |
| 13 | 3,5-dimethoxyphenyl 7-[(3S)- 3-methylpiperazin-1-yl]-1- benzofuran-5-sulfonate, trifluoroacetate | 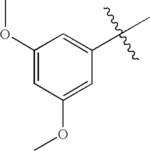 | 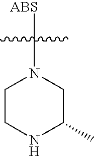 |
| 14 | 2-methoxy-5-methylphenyl 7- {[(3S)-3-methylpiperazin-1- yl]methyl}-1-benzofuran-5- sulfonate, bis(trifluoroacetate) | 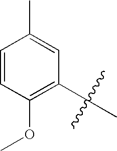 | 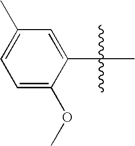 |
| 15 | 2-(aminocarbonyl)phenyl 7- {[(3S)-3-methylpiperazin-1- yl]methyl}-1-benzofuran-5- sulfonate, bis(trifluoroacetate) | 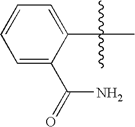 | 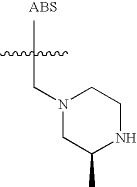 |
| 16 | 2-(aminocarbonyl)phenyl 7- {[(3R)-3-methylpiperazin-1- yl]methyl}-1-benzofuran-5- sulfonate, bis(trifluoroacetate) | 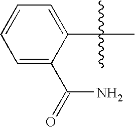 | 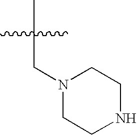 |
| 17 | 2-methoxy-5-methylphenyl 7- (piperazin-1-ylmethyl)-1- benzofuran-5-sulfonate, trifluoroacetate | 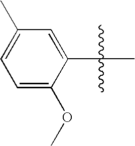 | 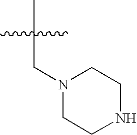 |
| 18 | 2-methoxy-5-methylphenyl 7- (1,4-diazepan-1-ylmethyl)-1- benzofuran-5-sulfonate, trifluoroacetate | 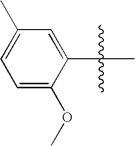 | 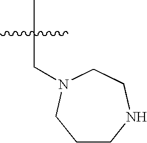 |

Methods

¹H nuclear magnetic resonance (NMR) and ¹³C NMR were recorded on a Bruker Advance DPX 400 spectrometer at 400.1 and 100.6 MHz, on a Varian Inova 400 instrument at 400 and 100,5 MHz respectively, or on a Bruker DRX 500 instrument at 500 and 125,7 MHz respectively. All spectra were recorded using residual solvent or tetramethylsilane (TMS) as internal standard. Electrospray mass spectrometry (MS) was performed using a Perkin-Elmer API 150EX mass spectrometer or an Agilent 1100 Series Liquid Chromatograph/Mass Selective Detector (MSD) to obtain the pseudo molecular [M+H]⁺ ion of the target molecules. Preparative HPLC/MS was performed on a Waters/Micromass Platform ZQ system equipped with System A: ACE 5 C8 column (19×50 mm), eluents: MilliQ water, MeCN and MilliQ/MeCN/0.1%TFA and system B: Xterra MS C18, 5 μm column (19×50 mm), eluents: MilliQ water, MeCN and NH₄HCO₃ (100 mM). Analytical HPLC was carried out on an Agilent Series 1100 system using either an ACE 3 C8 (3 μm, 3.0×50 mm) column (System A), a Chromolith SpeedROD RP-18e (4.6×50 mm) column (System B), or a YMC ODS-AQ (3 μm, 3.0×33 mm) column (System C). Acetonitrile and water containing 0.1% TFA were used as mobile phase for both analytical and preparative HPLC. Preparative flash chromatography was performed on Merck silica gel 60 (230-400 mesh). Microwave reactions were performed with a Personal Chemistry Smith Creator using 0.5-2 mL or 2-5 mL Smith Process Vials fitted with aluminum caps and septa. The compounds were named using ACD Name 6.0.

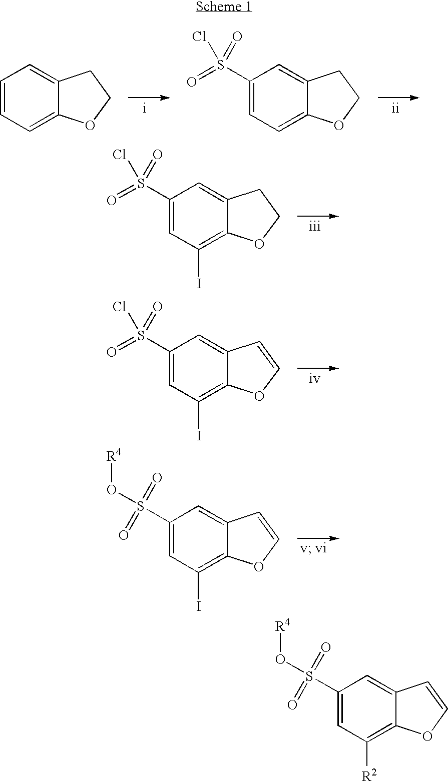

Legend to Scheme 1: i) Chlorosulfonic acid, DCM (dichloromethane), 5° C.→or t, ii) ICI, DCM, reflux temperature; iii) N-bromosuccinimide (NBS), azoisobutyronitrile (AIBN), chlorobenzene, 70° C.; iv) R⁴—OH, NaOH, benzyltrimethylammonium chloride, 40° C.; v) a secondary amine corresponding to Formula (XIII) or (XIV), or a protected derivative thereof, xylene, sodium tert-butoxide, Xantphos, tris(dibenzylideneacetone)di-palladium, 100-120° C.; and optionally vi) N-deprotection: HCl in diethyl ether.

Intermediate 1

### 2,3-Dihydro-benzofuran-5-sulfonyl chloride

Chlorosulphonic acid (43.4 g, 0.366 mol) in DCM (10 mL) was added to a cold solution (5° C.) of 2,3-dihydrobenzofuran (20 g, 0.166 mol) in DCM (200 mL). After the addition the reaction was left at room temperature over night. The reaction mixture was quenched with water (150 mL) keeping the temperature below 10° C. The organic phase was separated and washed with aqueous solution of NaHCO₃ (13,9 g in 150 mL of water). The organic solvents were evaporated giving a solid residue 3.3 g (23%). ¹H NMR 270 MHz (Chloroform-d) δ ppm 3.32 (t, J=8.91 Hz, 2 H) 4.75 (t, J=8.91 Hz, 2 H) 6.90 (d, J=9.15 Hz, 1 H) 7.78-7.90 (m, 2 H).

Intermediate 2

### 7-Iodo-2,3-dihydro-benzofuran-5-sulfonyl chloride

A solution of ICI (7.7 g, 47 mmol) in DCM (100 mL) was added drop wise to a solution of 2,3-dihydro-benzofuran-5-sulfonyl chloride (5 g, 23 mmol) in DCM (100 mL) under reflux temperature under nitrogen atmosphere. The reaction was heated to reflux temperature over night. The reaction was cooled at room temperature and acetonitrile (50 mL) was added. The reaction mixture was washed with a saturated solution of NaHCO₃ and the organic phase was separated followed by elimination of the volatile under vacuum to give 8 g of brown oil which was used to the next step without further purification. ¹H NMR 270 MHz (Chloroform-d) δ ppm 3.45 (t, J=8.91 Hz, 2 H) 4.82 (t, J=8.91 Hz, 2 H) 7.79 (d, J=1.48 Hz, 1 H) 8.16 (d,J=1.98 Hz, 1 H).

Intermediate 3

### 7-Iodo-benzofuran-5-sulfonyl chloride

AIBN (270 mg, 1.3 mmol) and NBS (2.5 g, 14 mmol) were added to (7-iodo-2,3-dihydro-benzofuran-5-sulfonyl chloride (4.4 g, 13 mmol) in chlorobenzene (30 mL) at 70 ° C. The heating was turned off one hour after the addition. Acetonitrile (30 mL) was added and the organic phase was washed with sodium sulphite in water. The organic phase was separated and the volatiles were evaporated to give 4 g of yellow crystals. ¹H NMR 270 MHz (Chloroform-d) δ ppm 7.07 (d, J=2.23 Hz, 1 H) 7.90 (d, J=2.23 Hz, 1 H) 8.29-8.37 (m, 1 H).

### EXAMPLE 1

### 2-Methoxy-5-methylphenyl 7-piperazin-1-yl-1-benzofuran-5-sulfonate, trifluoroacetate

The first synthetic step was performed according to the method described in the literature (J.Med.Chem. (2002), 45(5): 1086-1097). 7-Iodo-1benzofuran-5-sulfonyl chloride (0.095 g, 0.28 mmol; Intermediate 3) was dissolved in dichloromethane (5 mL) and then treated with 5-methyl-2-methoxyphenol (0.040 g, 0.29 mmol in 5 mL DCM), aqueous sodium hydroxide (5.0 M, 3 mL, 15 mmol) and benzyltrimethylammonium chloride (0.001 g, 0.01 mmol). The mixture was rapidly stirred at 40° C. After 16 h, dilution with DCM (30 mL) and water (10 mL) was performed. The layers were separated and the aqueous phase washed further with DCM (2×20 mL). The combined organic phase was washed with water (20 mL) and brine (20 mL) before drying over anhydrous magnesium sulfate. The solvent was removed under reduced pressure. The sample (0.087 g, 0.195 mmol) was dissolved in xylene (1.5 mL) at room temperature was treated with sodium tert-butoxide (0.029 g, 0.234 mmol), Xantphos (0.003 g, 0.005 mmol), tris(dibenzylideneacetone)dipalladium(0.004 g, 0.005 mmol) and t-BOC-piperazine (0.036 g, 0.195 mmol). The resulting suspension was heated to 100° C. for 16 h. On cooling, the mixture was filtered through celite eluting with xylene. The filtrate was concentrated under reduced pressure to give 110 mg of a brown oil. This material was dissolved in diethyl ether (2 mL) and treated with HCl (1 mL, 1.0 M in diethyl ether). After 16 h, the sample was concentrated under reduced pressure and then purified by prep HPLC to give 0.0055 g (3.8% over 3 steps). HPLC 91%, R_{T}=2.747 min (system A, 5-60% MeCN over 3 min); 95%, R_{T}=2.381 min (system B, 5-60% MeCN over 3min); ¹H NMR (270 MHz, METHANOL-D₄) δ ppm 2.23 (s, 3 H) 3.34 (s, 3 H) 3.43-3.50 (m, 4 H) 3.52-3.61 (m, 4 H) 6.78 (d, J=8.41 Hz, 1 H) 6.94-7.06 (m, 3 H) 7.21 (d, J=1.73 Hz, 1 H) 7.78 (d, J=1.73 Hz, 1 H) 7.96 (d, J=2.23 Hz, 1 H). MS (ESI+) for C₂₀H₂₂N₂O₅S m/z 403 (M+H).

### EXAMPLE 2

### 2-Chlorophenyl 7-piperazin-1-yl-1-benzofuran-5-sulfonate, trifluoroacetate

Prepared from 7-iodo-1benzofuran-5-sulfonyl chloride (0.09 g, 0.2 mmol; Intermediate 3) and 2-chlorophenol (0.03 g, 0.2 mmol) by the same method as Example 1 Yield: 0.0036 g (2.5% over 3 steps); HPLC 93%, R_{T}=2.755min (system A, 5-60% MeCN over 3 min); 100%, R_{T}=2.396min (system B, 5-60% MeCN over 3 min); ¹H NMR (270 MHz, METHANOL-D₄) δ ppm 3.38-3.52 (m, 4 H) 3.52-3.71 (m, 4 H) 7.01 (d, J=2.23 Hz, 1 H) 7.17-7.50 (m, 5 H) 7.84 (d, J=1.73 Hz, 1 H) 7.98 (d, J=2.23 Hz, 1 H); MS (ESI+) for C₁₈H₁₇ClN₂O₄S m/z 393 (M+H).

### EXAMPLE 3

### 2-(Trifluoromethyl)phenyl 7-piperazin-1-yl-1-benzofuran-5-sulfonate, trifluoroacetate

Prepared from 7-iodo-1benzofuran-5-sulfonyl chloride (0.095 g, 0.28 mmol; Intermediate 3) and 2-hydroxybenzotrifluoride (0.048 g, 0.29 mmol) by the same method as Example 1. Yield: 0.0031 g (2.1% over 3 steps); HPLC 92%, R_{T}=2.906 min (system A, 5-60% MeCN over 3 min); 97%, R_{T}=2.522 min (system B, 5-60% MeCN over 3 min); ¹H NMR (270 MHz, METHANOL-D₄) δ ppm 3.44-3.51 (m, 4 H) 3.58-3.66 (m, 4 H) 7.05 (d, J=2.23 Hz, 1 H) 7.34 (d, J=1.48 Hz, 1 H) 7.39-7.49 (m, J=7.55, 7.55 Hz, 1 H) 7.54-7.62 (m, 1 H) 7.62-7.73 (m, J=7.55, 7.55 Hz, 2 H) 7.94 (d, J=1.48 Hz, 1 H) 8.00 (d, J=2.23 Hz, 1 H); MS (ESI+) for C₁₉H₁₇F₃N₂O₄S m/z 427 (M+H).

Intermediate 4

### 2,3-Dimethoxyphenyl 7-iodo-1-benzofuran-5-sulfonate

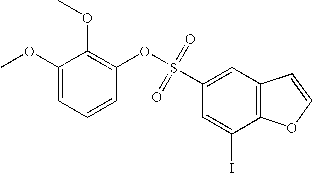

To a solution of Intermediate 2 (500 mg, 1 equiv) in chlorobenzene (10 mL) stirred at 80° C. was added AIBN (42 mg, 0.15 equiv), followed by NBS (285 mg, 1.1 equiv) with continued stirring at 80° C. for 120 min. The reaction mixture was chilled with ice water, and then filtered with a filter tube to remove solid succinimide. To the filtrate was added 2,3-dimethoxy phenol (227 μL, 1.2 equiv), followed by pyridine (376 μL, 3.2 equiv). The resulting mixture was stirred at 50° C. for 20 h. Then the heating was increased to 80° C. during 120 min to drive reaction towards product. The reaction mixture was diluted with (50 mL) EtOAc and washed with 1M HCl (25 mL), followed by water (25 ml) and brine (25 mL), dried Na₂SO₄ and evaporated to give 818.8 mg. Purified by column chromatography (SiO₂: p-ether:ether, 4:1) to give 184.1 mg (28% yield): HPLC 90%, R_{T}=2.64 min (System A, 10-97% MeCN over 3 min), 90%, R_{T}=2.64 min (System C, 10-97% MeCN over 3 min); ¹H NMR (400 MHz, CHLOROFORM-D) δ ppm 3.70 (s, 3 H) 3.81 (s, 3 H) 6.75-6.84 (m, 2 H) 6.94-7.01 (m, 2 H) 7.82 (d, J=2.20 Hz, 1 H) 8.16 (d, J=1.46 Hz, 1 H) 8.25 (d, J=1.71 Hz, 1 H); MS (ESI+) for C₁₆H₁₃IO₆S m/z 461 (M+H)⁺.

Intermediate 5

### Pyridin-3-yl 7-iodo-1-benzofuran-5-sulfonate

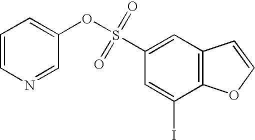

The title compound was prepared according to the method described for Intermediate 4 from Intermediate 2, to give the desired product (220.9 mg, 47% yield): HPLC 95%, R_{T}=2.27 min (System A, 10-97% MeCN over 3 min); MS (ESI+) for C₁₃H₈INO₄S m/z 402 (M+H)⁺.

### EXAMPLE 4

### Pyridin-3-yl 7-piperazin-1-yl-1-benzofuran-5-sulfonate hydrochloride

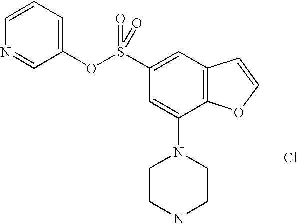

t-BOC-piperazine (23 mg, 1 equiv), sodium tert-butoxide (14 mg, 1.2 equiv), Pd₂(dba)₃ (5 mg, 0.04 equiv), Xantphos (3 mg, 0.04 equiv) were added to a reaction tube and flushed with N₂. Intermediate 5 (50 mg, 1 equiv) in (3 mL) xylene was added and the reaction mixture stirred at 120° C. for 4 hrs. The reaction mixture was allowed to cool to rt and then filtered through Celite, eluting with xylene. The filtrate was evaporated to give 40 mg as a pale yellow oil. The residue was purified by Prep LCMS and the pure fractions evaporated. Redissolved in MeOH and added 1 M HCl in diethyl ether to deprotect (i.e., to cleave off the t-BOC group) and convert into HCl-salt, evaporated to give 14.9 mg (28% yield) of the title product as a tan solid: HPLC 93%, R_{T}=1.49 min (System A, 10-97% MeCN over 3 min), 94%, R_{T}=1.35 min (System C, 10-97% MeCN over 3 min); ¹H NMR (400 MHz, METHANOL-D₄) δ ppm 0.80-0.99 (m, 2 H) 1.18-1.47 (m, 3 H) 3.48 (s, 2 H) 3.59-3.76 (m, 2 H) 4.20 (t, J=5.62 Hz, 1 H) 7.02 (s, 1 H) 7.31 (s, 1 H) 7.60 (dd, J=5.25, 3.54 Hz, 1 H) 7.65-7.74 (dd, J=5.25, 3.30 Hz, 1 H) 7.93 (m, 2 H) 8.77 (s, 1 H); MS (ESI+) for C₁₇H₁₇N₃O₄S m/z 360 (M+H)⁺.

Intermediate 6

### 2-methoxy-5-methylphenyl 7-iodo-1-benzofuran-5-sulfonate

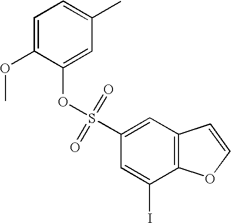

A solution of 7-iodo-2,3-dihydro-benzofuran-5-sulfonyl chloride (1024 mg, 2.97 mmol; Intermediate 2), NBS (609 mg, 3.42 mmol) and AIBN (57 mg, 0.35 mmol) in chlorobenzene (20 mL) was heated at 80° C. for 2 h. The reaction mixture was allowed to cool and filtered. The solvent was evaporated and the crude product was dissolved in CH₂Cl₂ (5 mL). A solution of 2-methoxy-5-methylphenol (529 mg, 3.83 mmol) in CH₂Cl₂ (6 mL) was added followed by triethylamine (525 μL, 3.78 mmol). The reaction mixture was stirred at room temperature for 2 h. The solvent was evaporated and the material was used in other experiments without further purification or characterization.

Intermediate 7

### 2-Methoxy-5-methylphenyl 7-cyano-1benzofuran-5-sulfonate

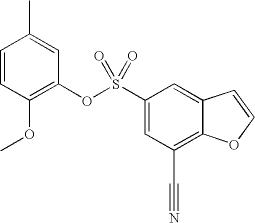

A reaction mixture of Intermediate 6 (1.39 g, 3.13 mmol), Zn(CN)₂ (0.92 g, 7.82 mmol) and Pd(PPh₃)₄ (0.43 g, 0.37 mmol) in DMF (14 mL) was exposed to microwave irradiation for 20 minutes at 180° C. The mixture was centrifuged and the solvent was poured off from the solid The solvent was evaporated and the residue was chromatographed on SiO₂ eluting with (DCM: p-ether, 1:1) giving (0.91 g, 2.66 mmol, yield 85%) solid material. ¹H NMR (400 MHz, CHLOROFORM-D) δ ppm 2.29 (s, 3 H) 3.44 (s, 3 H) 6.67-6.72 (m, 1 H) 6.99-7.03 (m, J=2.44 Hz, 2 H) 7.07-7.10 (m, 1 H) 7.90-7.93 (m, 1 H) 8.16-8.18 (m, 1 H) 8.39-8.42 (m, 1 H). HPLC 100%, R_{T}=2.34 min (System A, 30-80% MeCN over 3 min), 100%, R_{T}=2.38 min (System C, 30-80% MeCN over 3 min). MS (ESI⁺) for C₁₇H₁₃NO₅S m/z 343 (M+H)⁺.

Intermediate 8

### 2-Methoxy-5-methylphenyl 7-formyl-1-benzofuran-5-sulfonate

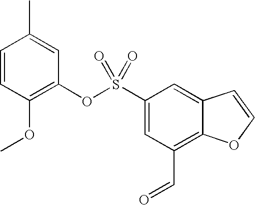

A suspension of Intermediate 7 (0.91 g, 2.65 mmol) and PtO₂ (60 mg) in a solvent mixture of 80% HCOOH in H₂O (50 mL)/THF (20 mL) was stirred at 60° C. Additionally PtO₂ (20 mg) was repeatedly added every 30 minute during the reaction time. After 8 h was the solvent evaporated and the residue was chromatographed on SiO₂ eluting with (DCM: p-ether, 7: 3) giving (0.57 g, 1.64 mmol, yield 62%) solid material.

¹H NMR (400 MHz, CHLOROFORM-D) δ ppm 2.27 (s, 3 H) 3.42 (s, 3 H) 6.65-6.70 (m, 1 H) 6.96-7.01 (m, 2 H) 7.05-7.09 (m, 1 H) 7.90-7.94 (m, 1 H) 8.37-8.44 (m, 2 H) 10.44 (s, 1 H). HPLC 94%, R_{T}=2.41 min (System A, 10-97% MeCN over 3 min), 93%, R_{T}=2.40 min (System C, 10-97% MeCN over 3 min). MS (ESI⁺) for C₁₇H₁₄NO₆S m/z 347 (M+H)⁺.

### EXAMPLE 5

### 2-Methoxy-5-methylphenyl 7-[(4-methylpiperazin-1-yl)methyl]-1-benzofuran-5-sulfonate bis(trifluoroacetate)

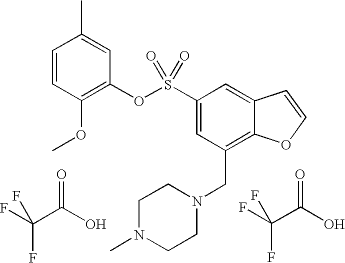

A reaction mixture of Intermediate 8 (40 mg, 0.11 mmol), sodium triacetoxyborohydride (73 mg, 0.35 mmol), acetic acid (66 uL, 1.15 mmol) and 1-methylpiperazine (35 mg, 0.35 mmol) in THF (1.5 mL) was exposed to microwave irradiation for 12 minutes at 130° C. The solvent was evaporated and the residue was purified with preparative LC-MS giving the title compound as a solid (43 mg, 0.065 mmol, yield 59%). ¹H NMR (400 MHz, METHANOL-D₄) δ ppm 2.24 (s, 3 H) 2.68-2.87 (m, 4 H) 2.89 (s, 3 H) 3.24-3.30 (m, 4 H) 3.31-3.32 (m, 3 H) 4.03 (s, 2 H) 6.74-6.77 (m, 1 H) 6.99-7.03 (m, 2 H) 7.04-7.06 (m, 1 H) 7.76-7.78 (m, 1 H) 7.98-7.99 (m, 1 H) 8.11-8.13 (m, 1 H). HPLC 97%, R_{T}=1.93 min (System A, 10-97% MeCN over 3 min), 100%, R_{T}=1.76 min (System C, 10-97% MeCN over 3 min). MS (ESI⁺) for C₂₂H₂₆N₂O₅S m/z 431 (M+H)⁺.

### EXAMPLE 6

### 2-Methoxy-5-methylphenyl 7-{[(3R)-3-methylpiperazin-1-yl]methyl}-1-benzofuran-5-sulfonate bis(trifluoroacetate)

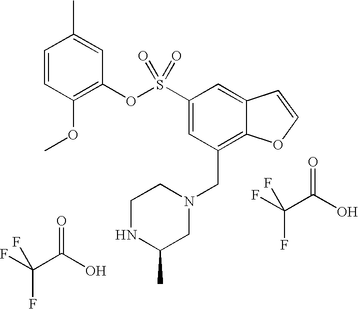

The synthesis of the title compound was performed using the method described for Example 5 with Intermediate 8 (30 mg, 0.087 mmol), sodium triacetoxyborohydride (55 mg, 0.26 mmol), acetic acid (49 uL, 0.86 mmol) and R-2-methylpiperazine (26 mg, 0.26 mmol) in THF (1.5 mL) giving the title compound as an oil (15 mg, 0.023 mmol, yield 26 %). ¹H NMR (400 MHz, METHANOL-D₄) δ ppm 1.28 (d, J=6.84 Hz, 3 H) 2.24 (s, 3 H) 2.29-2.37 (m, 1 H) 2.43-2.53 (m, 1 H) 2.91-2.99 (m, 1 H) 3.03-3.18 (m, 2 H) 3.33 (s, 3 H) 3.35-3.42 (m, 2 H) 4.00-4.05 (d, J=5.62 Hz, 2 H) 6.74-6.79 (m, 1 H) 6.98-7.03 (m, 2 H) 7.05-7.06 (m, 1 H) 7.79-7.80 (m, 1 H) 7.98-8.00 (m, 1 H) 8.11-8.13 (m, 1 H). HPLC 100%, R_{T}=1.90 min (System A, 10-97% MeCN over 3 min), 100%, R_{T}=1.72 min (System C, 10-97% MeCN over 3 min). MS (ESI⁺) for C₂₂H₂₆N₂O₅S m/z 431 (M+H)⁺.

### EXAMPLE 7

### Pyridin-3-yl 7-(4-methylpiperazin-1-yl)-1-benzofuran-5-sulfonate trifluoroacetate

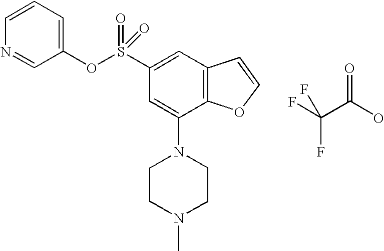

The title compound was prepared according to a method similar to that described for Example 4, except the deprotection step, from Intermediate 5 and N-methylpiperazine, to give the desired product (1.9 mg, 8% yield) as a colorless gum: HPLC 100%, R_{T}=1.53 min (System A, 10-97% MeCN over 3 min), 100%, R_{T}=1.39 min (System C, 10-97% MeCN over 3 min); ¹H NMR (400 MHz, METHANOL-D₄) δ ppm 3.00 (s, 3 H) 3.41-3.86 (m, 4 H) 4.05-4.14 (m, 2 H) 4.24-4.36 (m, 2 H) 7.02 (d, J=2.20 Hz, 1 H) 7.24 (d, J=1.46 Hz, 1 H) 7.43 (dd, J=8.42, 4.76 Hz, 1 H) 7.51-7.67 (m, 1 H) 7.81 (d, J=1.71 Hz, 1 H) 7.99 (d, J=2.20 Hz, 1 H) 8.05-8.21 (m, 1 H) 8.45 (d, J=4.64 Hz, 1 H); MS (ESI+) for C₁₈H₁₉N₃O₄S m/z 374 (M+H)⁺.

### EXAMPLE 8

### 2,3-Dimethoxyphenyl 7-(4-methylpiperazin-1-yl)-1-benzofuran-5-sulfonate trifluoroacetate

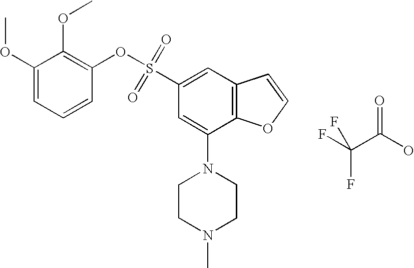

The title compound was prepared according to a method similar to that described for Example 4, except the deprotection step, from Intermediate 4 and N-methylpiperazine, to give the desired product (3.0 mg, 11% yield) as a light yellow gum: HPLC 90%, R_{T}=1.89 min (System A, 10-97% MeCN over 3 min), 92%, R_{T}=1.73 min (System C, 10-97% MeCN over 3 min); ¹H NMR (400 MHz, METHANOL-D₄) δ ppm 3.01 (s, 3 H) 3.24 (s, 2 H) 3.43 (s, 2 H) 3.63 (s, 3 H) 3.67 (s, 2 H) 3.80 (s, 3 H) 4.06 (s, 2 H) 6.70 (dd, J=8.06, 1.71 Hz, 1 H) 6.90-7.04 (m, 3 H) 7.30 (d, J=1.46 Hz, 1 H) 7.86 (d, J=1.71 Hz, 1 H) 7.96 (d, J=2.20 Hz, 1 H); MS (ESI+) for C₂₁H₂₄N₂O₆S m/z 433 (M+H)⁺.

### EXAMPLE 9

### 2,3-Dimethoxyphenyl 7-[(3R)-3-methylpiperazin-1-yl]-1-benzofuran-5-sulfonate trifluoroacetate

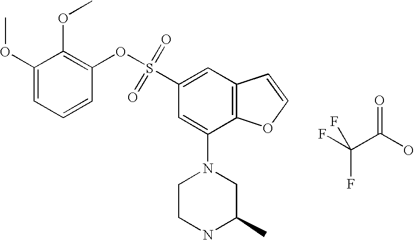

The title compound was prepared according to a method similar to that described for Example 4, except the deprotection step, from Intermediate 4 and (2R)-2-methylpiperazine, to give the desired product (5.2 mg, 19% yield) as a dark brown gum: HPLC 97%, R_{T}=1.91 min (System A, 10-97% MeCN over 3 min), 97%, R_{T}=1.74 min (System C, 10-97% MeCN over 3 min); ¹H NMR (400 MHz, METHANOL-D₄) δ ppm 1.43 (d, J=6.59 Hz, 3 H) 3.00 (dd, J=13.18, 10.25 Hz, 1 H) 3.15-3.24 (m, 1 H) 3.37-3.47 (m, 1 H) 3.52-3.59 (m, 2 H) 3.63 (s, 3 H) 3.80 (s, 3 H) 3.91-4.02 (m, 2 H) 6.70 (dd, J=8.06, 1.71 Hz, 1 H) 6.90-7.04 (m, 3 H) 7.29 (d, J=1.22 Hz, 1 H) 7.85 (d, J=1.46 Hz, 1 H) 7.96 (d, J=2.20 Hz, 1 H); MS (ESI+) for C₂₁H₂₄N₂O₆S m/z 433 (M+H)⁺.

### EXAMPLE 10

### 2,3-Dimethoxyphenyl 7-[(3S)-3-methylpiperazin-1-yl]-1-benzofuran-5-sulfonate trifluoroacetate

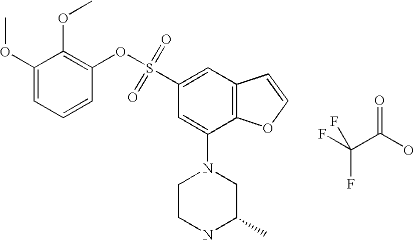

The title compound was prepared according to a method similar to that described for Example 4, except the deprotection step, from Intermediate 4 and (2S)-2-methylpiperazine, to give the desired product (2.6 mg, 10% yield) as a brown oil: HPLC 100%, R_{T}=1.91 min (System A, 10-97% MeCN over 3 min), 100%, R_{T}=1.76 min (System C, 10-97% MeCN over 3 min); ¹H NMR (400 MHz, METHANOL-D₄) δ ppm 1.43 (d, J=6.59 Hz, 3 H) 3.00 (dd, J=13.18, 10.25 Hz, 1 H) 3.12-3.26 (m, 1 H) 3.33-3.49 (m, 1 H) 3.52-3.63 (m, 2 H) 3.59-3.67 (m, 3 H) 3.76-3.83 (m, 3 H) 3.89-4.05 (m, 2 H) 6.70 (dd, J=8.06, 1.71 Hz, 1 H) 6.87-7.07 (m, 3 H) 7.30 (d, J=1.46 Hz, 1 H) 7.85 (d, J=1.71 Hz, 1 H) 7.96 (d, J=1.95 Hz, 1 H); MS (ESI+) for C₂₁H₂₄N₂O₆S m/z 433 (M+H)⁺.

Intermediate 9

### 3,5-Dimethoxyphenyl 7-iodo-1-benzofuran-5-sulfonate

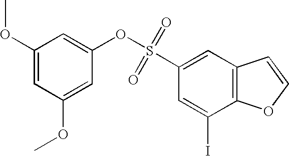

The title compound was prepared according to a method similar to that described for Intermediate 4 from Intermediate 3, to give the desired product (159.7 mg, 30% yield): HPLC 65%, R_{T}=2.67 min (System A, 10-97% MeCN over 3 min); MS (ESI+) for C₁₆H₁₃IO₆S m/z 461 (M+H)⁺.

### EXAMPLE 11

### 3,5-Dimethoxyphenyl 7-(4-methylpiperazin-1-yl)-1-benzofuran-5-sulfonate trifluoroacetate

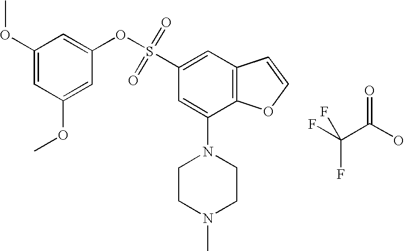

The title compound was prepared according to a method similar to that described for Example 4, except the deprotection step, from Intermediate 9 and N-methylpiperazine, to give the desired product (1.6 mg, 6% yield) as a light brown gum: HPLC 100%, R_{T}=1.96 min (System A, 10-97% MeCN over 3 min), 100%, R_{T}=1.81 min (System C, 10-97% MeCN over 3 min); ¹H NMR (400 MHz, METHANOL-D₄) δ ppm 3.01 (s, 3 H) 3.10-3.22 (m, 2 H) 3.37-3.49 (m, 2 H) 3.63 (s, 6 H) 3.66-3.82 (m, 2 H) 3.97-4.14 (m, 2 H) 6.10 (d, J=2.20 Hz, 2 H) 6.35-6.39 (m, 1 H) 7.03 (d, J=2.20 Hz, 1 H) 7.22 (d, J=1.46 Hz, 1 H) 7.84 (d, J=1.71 Hz, 1 H) 7.98 (d, J=1.95 Hz, 1 H); MS (ESI+) for C₂₁H₂₄N₂O₆S m/z 4.33 (M+H)⁺.

### EXAMPLE 12

### 3,5-Dimethoxyphenyl 7-[(3R)-3-methylpiperazin-1-yl]-1-benzofuran-5-sulfonate trifluoroacetate

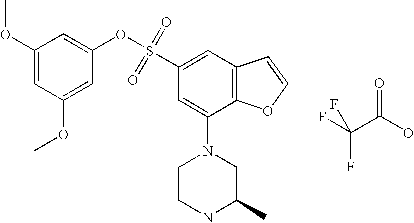

The title compound was prepared according to a method similar to that described for Example 4, except the deprotection step, from Intermediate 9 and (2R)-2-methylpiperazine, to give the desired product (1.3 mg, 4% yield) as a light brown gum: HPLC 100%, R_{T}=1.99 min (System A, 10-97% MeCN over 3 min), 100%, R_{T}=1.84 min (System C, 10-97% MeCN over 3 min); ¹H NMR (400 MHz, METHANOL-D₄) δ ppm 1.43 (d, J=6.59 Hz, 3 H) 3.19 (d, J=10.50 Hz, 1 H) 3.36-3.49 (m, 2 H) 3.52-3.58 (m, 2 H) 3.63 (s, 6 H) 3.96 (s, 2 H) 6.11 (d, J=2.20 Hz, 2 H) 6.38 (s, 1 H) 7.03 (d, J=2.20 Hz, 1 H) 7.21 (d, J=1.46 Hz, 1 H) 7.83 (d, J=1.47 Hz, 1 H) 7.97 (d, J=2.20 Hz, 1 H); MS (ESI+) for C₂₁H₂₄N₂O₆S m/z 433 (M+H)⁺.

### EXAMPLE 13

### 3,5-Dimethoxyphenyl 7-[(3S)-3-methylpiperazin-1-yl]-1-benzofuran-5-sulfonate trifluoroacetate

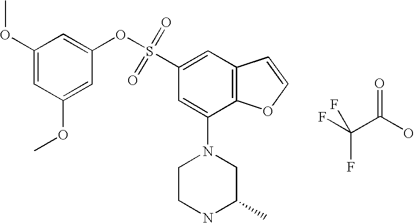

The title compound was prepared according to a method similar to that described for Example 4, except the deprotection step, from Intermediate 9 and (2S)-2-methylpiperazine, to give the desired product (1.3 mg, 4% yield) as a light brown gum: HPLC 100%, R_{T}=1.99 min (System A, 10-97% MeCN over 3 min), 100%, R_{T}=1.83 min (System C, 10-97% MeCN over 3 min); ¹H NMR (400 MHz, METHANOL-D₄) δ ppm 1.39-1.46 (m, J=6.59 Hz, 3 H) 2.93-3.03 (m, 1 H) 3.11-3.23 (m, 1 H) 3.36-3.47 (m, 2 H) 3.52-3.59 (m, 1 H) 3.63 (s, 6 H) 3.91-4.03 (m, 2 H) 6.10 (d, J=2.20 Hz, 2 H) 6.38 (t, J=2.08 Hz, 1H) 7.03 (d, J=2.20 Hz, 1 H) 7.21 (d, J=1.46 Hz, 1 H) 7.83 (d, J=1.46 Hz, 1 H) 7.97 (d, J=2.20 Hz, 1 H); MS (ESI+) for C₂₁H₂₄N₂O₆S m/z 433 (M+H)⁺.

### EXAMPLE 14

### 2-Methoxy-5-methylphenyl 7-{[(3S)-3-methylpiperazin-1-yl]methyl}-1-benzofuran-5-sulfonate bis(trifluoroacetate)

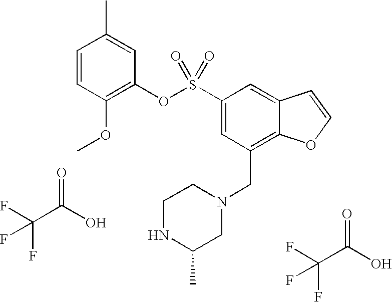

The synthesis of the title compound was performed using the method described for Example 5 with Intermediate 8 (30 mg, 0.087 mmol), sodium triacetoxyborohydride (55 mg, 0.26 mmol), acetic acid (49 uL, 0.86 mmol) and S-2-methylpiperazine (26 mg, 0.26 mmol) in THF (1.5 mL) giving (24 mg, 0.036, yield 41%) oil. ¹H NMR (400 MHz, METHANOL-D₄) δ ppm 1.29 (d, J=6.59 Hz, 3 H) 2.23 (s, 3 H) 2.34-2.42 (m, 1 H) 2.49-2.58 (m, 1 H) 2.96-3.02 (m, 1 H) 3.07-3.19 (m, 2 H) 3.33 (s, 3 H) 3.34-3.44 (m, 2 H) 4.07 (d, J=5.62 Hz, 2 H) 6.76 (d, J=8.30 Hz, 1 H) 6.98-7.03 (m, 2 H) 7.06 (d, J=2.20 Hz, 1 H) 7.80-7.81 (m, 1 H) 7.98-8.00 (m, 1 H) 8.12-8.13 (m, 1 H). HPLC 97%, R_{T}=1.90 min (System A, 10-97% MeCN over 3 min), 100%, R_{T}=1.73 min (System C, 10-97% MeCN over 3 min). MS (ESI+) for C₂₂H₂₆N₂O₅S m/z 431 (M+H)⁺.

Intermediate 10

### 2-(Aminocarbonyl)phenyl 7-iodo-1-benzofuran-5-sulfonate

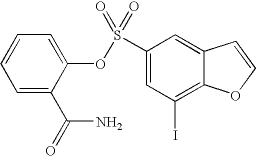

A mixture of intermediate 2 (3.0 g, 8.70 mmol), NBS (1.78 g, 10.01 mmol) and AIBN (0.17 g, 1.04 mmol) in chlorobenzene (40 mL) was stirred at 80° C. for 2 h. The mixture was chilled to room temperature and solid material was filtered off. The solvent was evaporated and the residue was dissolved in DCM (25 mL). A solution of salicylamide (1.55 g, 11.32 mmol) in DCM (80 mL) was added followed by triethylamine (1.56 mL, 11.32 mmol) and the mixture was stirred over night. The mixture was diluted with DCM (50 mL) and washed with 1M NaOH (50 mL). The organic phase was separated and dried over Na₂SO₄. The solid was filtered off and the solvent was evaporated giving a solid. The dark solid was triturated twice with DCM (10 mL) giving (2.27 g, 5.12 mmol, yield 51%) the title compound as a white solid. ¹H NMR (400 MHz, DMSO-D₆) δ ppm 7.10-7.13 (m, 1 H) 7.28-7.30 (m, 1 H) 7.34-7.54 (m, 4 H) 7.59-7.63 (m, 1 H) 8.04-8.06 (m, 1 H) 8.22-8.24 (m, 1 H) 8.31-8.33 (m, 1 H). HPLC 82%, R_{T}=2.06 min (System A, 10-97% MeCN over 3 min), 83%, R_{T}=1.98 min (System C, 10-97% MeCN over 3 min). MS (ESI⁺) for C₁₅H₁₀INO₅S m/z 444 (M+H)⁺.

Intermediate 11

### 2-(Aminocarbonyl)phenyl 7-vinyl-1-benzofuran-5-sulfonate

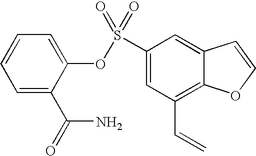

A mixture of intermediate 10 (1.61 g, 3.64 mmol), tributyl(vinyl)tin (2.31 g, 7.28 mmol) and Pd(PPh₃)₂OAc₂ (0.27 g, 0.26 mmol) was exposed to microwave irradiation for 20 minutes at 160° C. The solvent was evaporated and the residue was chromatographed on SiO₂ eluting with CHCl₃/p-ether (7:3) to give (867 mg, 2.52 mmol, yield 69%) the title compound as a solid. ¹H NMR (400 MHz, CHLOROFORM-D) δ ppm 5.64 (dd, J=11.23, 0.73 Hz, 1 H) 5.69-5.78 (m, 1 H) 6.24 (dd, J=17.70, 0.85 Hz, 1 H) 6.59-6.66 (m, 1 H) 6.88 (d, J=2.20 Hz, 1 H) 6.90-6.99 (m, 1 H) 7.13-7.16 (m, 1 H) 7.31-7.36 (m, 1 H) 7.39-7.42 (m, 1 H) 7.79-7.82 (m, 2 H) 7.88-7.91 (m, 1 H) 8.01-8.02 (m, 1 H). HPLC 90% R_{T}=2.09 min (System A, 10-97% MeCN over 3 min), 91%, R_{T}=2.00 min (System C, 10-97% MeCN over 3 min). MS (ESI+) for C₁₇H₁₃NO₅S m/z 344 (M+H)⁺.

Intermediate 12

### 2-(Aminocarbonyl)phenyl 7-formyl-1-benzofuran-5-sulfonate

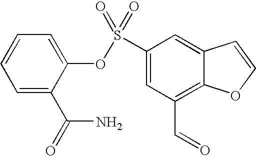

To a solution of Intermediate 11 (0.77 g, 2.25 mmol) in 25% H₂O in dioxane (40 mL) was 2,6-lutidine (0.48 g, 4.51 mmol), OSO₄ (11 mg, 0.045 mmol) and NaIO₄ (1.93 g, 9.01 mmol) added and the mixture was stirred for 2 h. Water was added and the mixture was extracted with CHCl₃. The organic phase was dried over MgSO₄, filtered and the solvent was evaporated. The residue was chromatographed on SiO₂ eluting with CHCl₃/acetone (8:2) to give (0.25 g, 0.73 mmol, yield 33%) the title compound as a white solid. ¹H NMR (400 MHz, CHLOROFORM-D) δ ppm 5.62-5.71 (m, 1 H) 6.42-6.51 (m, 1 H) 6.97-7.00 (m, 1 H) 7.26-7.28 (m, 1 H) 7.32-7.39 (m, 1 H) 7.44-7.50 (m, 1 H) 7.79-7.83 (m, 1 H) 7.92-7.95 (m, 1 H) 8.29-8.34 (m, 2 H) 10.47 (s, 1 H). HPLC 100%, R_{T}=1.92 min (System A, 10-97% MeCN over 3 min), 97%, R_{T}=2.52 min (System C, 10-97% MeCN over 3 min). MS (ESI⁺) for C₁₇H₁₁NO₆S m/z 346 (M+H)⁺.

### EXAMPLE 15

### 2-(Aminocarbonyl)phenyl 7-{[(3S)-3-methylpiperazin-1-yl]methyl}-1benzofuran-5-sulfonate bis(trifluoroacetate)

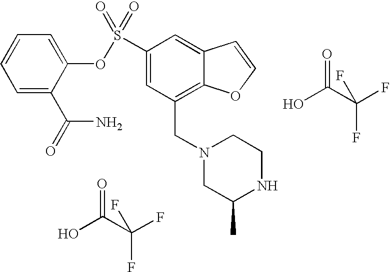

The synthesis of the title compound was performed using the method described for Example 5 with Intermediate 12 (40 mg, 0.11 mmol), sodium triacetoxyborohydride (73 mg, 0.35 mmol), acetic acid (66 uL, 1.15 mmol) and R-2-methylpiperazine (35 mg, 0.35 mmol) in THF (1.5 mL) giving (42 mg, 0.064, yield 58%). ¹H NMR (400 MHz, METHANOL-D₄) δ ppm 1.30 (d, J=6.59 Hz, 3 H) 2.35-2.44 (m, 1 H) 2.52-2.62 (m, 1 H) 3.05-3.24 (m, 3 H) 3.35-3.49 (m, 2 H) 4.10 (s, 2 H) 7.02-7.05 (m, 1 H) 7.32-7.38 (m, 2 H) 7.47-7.56 (m, 2 H) 7.78-7.81 (m, 1 H) 7.98-8.01 (m, 1 H) 8.12-8.14 (m, 1 H). HPLC 100%, R_{T}=1.58 min (System A, 10-97% MeCN over 3 min), 100%, R_{T}=2.10 min (System C, 10-97% MeCN over 3 min). MS (ESI⁺) for C₂₁H₂₃N₃O₅S m/z 330 (M+H)⁺.

### EXAMPLE 16

### 2-(Aminocarbonyl)phenyl 7-{[(3R)-3-methylpiperazin-1-yl]methyl}-1-benzofuran-5-sulfonate bis(trifluoroacetate)

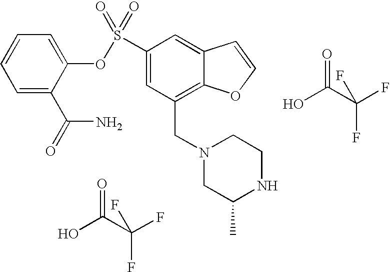

The synthesis of the title compound was performed using the method described for Example 5 with Intermediate 12 (40 mg, 0.11 mmol), sodium triacetoxyborohydride (73 mg, 0.35 mmol), acetic acid (66 μL, 1.15 mmol) and S-2-methylpiperazine (35 mg, 0.35 mmol) in THF (1.5 mL) giving (23 mg, 0.035, yield 32%). ¹H NMR (400 MHz, METHANOL-D₄) δ ppm 1.30 (d, J=6.59 Hz, 3 H) 2.37-2.45 (m, 1 H) 2.54-2.63 (m, 1 H) 3.06-3.25 (m, 3 H) 3.36-3.49 (m, 2 H) 4.11 (s, 2 H) 7.02-7.05 (m, 1 H) 7.32-7.39 (m, 2 H) 7.48-7.55 (m, 2 H) 7.79-7.81 (m, 1 H) 7.98-8.01 (m, 1 H) 8.12-8.15 (m, 1 H). HPLC 100%, R_{T}=1.58 min (System A, 10-97% MeCN over 3 min), 100%, R_{T}=2.10 min (System C, 10-97% MeCN over 3 min). MS (ESI^{+) for C}₂₁H₂₃ N₃O₅S m/z 330 (M+H)⁺.

### EXAMPLE 17

### 2-Methoxy-5-methylphenyl 7-(piperazin-1-ylmethyl)-1-benzofuran-5-sulfonate trifluoroacetate

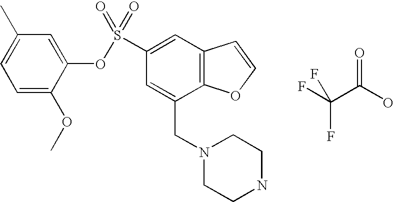

To Intermediate 8 (250 mg, 1 equiv) in (5 mL) THF was added t-BOC-piperazine (404 mg, 3 equiv) and the reaction mixture stirred at rt for 20 min. Acetic acid (413 μL, 10 equiv) and sodium triacetoxyborohydride (460 mg, 3 equiv) were added and the reaction mixture stirred at rt overnight. The reaction mixture was filtered and the filtrate evaporated to give 462 mg as a pale yellow oil. The residue was taken up in DCM (10 mL) and TFA (1 mL) was added and stirred at rt overnight to deprotect. The residue was purified by Prep LCMS and the pure fractions evaporated to give 167.9 mg (44% yield) as a white solid: HPLC 100%, R_{T}=1.76 min (System A, 10-97% MeCN over 3 min), 100%, R_{T}=1.61 min (System C, 10-97% MeCN over 3 min); ¹H NMR (400 MHz, METHANOL-D₄) δ ppm 2.23 (s, 3 H) 2.77-2.85 (m, 4 H) 3.20-3.26 (m, 4 H) 3.27-3.29 (m, 3 H) 4.07 (s, 2 H) 6.70-6.77 (m, 1 H) 6.96-7.03 (m, 2 H) 7.05 (d, J=2.20 Hz, 1 H) 7.77 (s, 1 H) 7.99 (d, J=2.20 Hz, 1 H) 8.13 (d, J=1.71 Hz, 1 H); MS (ESI+) for C₂₁H₂₄N₂O₅S m/z 417 (M+H)⁺.

### EXAMPLE 18

### 2-Methoxy-5-methylphenyl 7-(1,4-diazepan-1-ylmethyl)-1-benzofuran-5-sulfonate trifluoroacetate

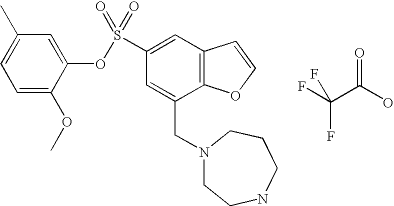

The title compound was prepared according to a method similar to that as described for Example 17 from Intermediate 8 and N-t-BOC-homopiperazine, to give the desired product (15.1 mg, 20% yield) as a white solid. The lower yield is due to the fact that a major amount of the sample was lost during workup: HPLC 100%, R_{T}=1.64 min (System A, 10-97% MeCN over 3 min), 100%, R_{T}=1.51 min (System C, 10-97% MeCN over 3 min); ¹H NMR (400 MHz, METHANOL-D₄) δ ppm 2.05-2.15 (m, 2 H) 2.24 (s, 3 H) 3.08-3.15 (m, 2 H) 3.32-3.38 (m, 7 H) 3.39-3.45 (m, 2 H) 4.44 (s, 2 H) 6.77 (d, J=8.30 Hz, 1 H) 6.97-7.05 (m, 2 H) 7.10 (d, J=2.20 Hz, 1 H) 7.93 (d, J=1.47 Hz, 1 H) 8.04 (d, J=2.20 Hz, 1 H) 8.20 (d, J=1.71 Hz, 1 H); MS (ESI+) for C₂₂H₂₆N₂O₅S m/z 431 (M+H)⁺.

Biological Tests

The ability of a compound according to the invention to bind to a 5-HT₆ receptor, and to be pharmaceutically useful, can be determined using in vivo and in vitro assays known in the art.

(a) 5-HT₆ Receptor Binding Assay

Binding affinity experiment for the human 5-HT6 receptor are performed in HEK293 cells transfected with 5-HT₆ receptor using (³H)-LSD as labeled ligand according to the general method as described by Boess F. G et al. Neuropharmacology vol. 36(4/5) 713-720, 1997.

Materials

Cell culture

The HEK-293 cell line transfected with the human 5-HT₆ receptor was cultured in Dulbeccos Modified Eagles Medium containing 5% dialyzed foetal bovine serum, (Gibco BRL 10106-169), 0.5 mM sodium pyruvate and 400 μg/ml Geneticin (G-418) (Gibco BRL 10131-019). The cells were passaged 1:10, twice a week.

Chemicals

The radioligand [³H] LSD 60-240 Ci/mmol, obtained from Amersham Pharmacia Biotech, (Buckinghamshire, England) was in ethanol and stored at −20° C. The unlabelled ligands, representing different selectivity profiles, are presented in Table 1. The compounds were dissolved in 100% DMSO and diluted with binding buffer.

Disposable Compounds were diluted in Costar 96 well V-bottom polypropylene plates (Corning Inc. Costar, N.Y., USA). Samples were incubated in Packard Optiplate (Packard Instruments B. V., Groningen, The Netherlands). The total amount of added radioligand was measured in Packard 24-well Barex plates (Packard Instruments B. V., Groningen, The Netherlands) in the presence of Microscint™ 20 scintillation fluid (Packard Bioscience, Meriden, Conn., USA).

Buffer

The binding buffer consisted of 20 mM HEPES, 150 mM NaCl, 10 mM MgCl₂, and 1 mM, EDTA, pH 7.4.

Methods

Membrane Preparation

Cells were grown to approximately 90% confluence on 24.5×24.5 NUNC culture dishes. The medium was aspirated, and after rinsing with ice-cold PBS, the cells were scraped off using 25 ml Tris buffer (50 mM Tris-HCl, 1 mM EDTA, 1 mM EGTA, pH 7.4) and a window scraper. The cells were then broken with a Polytron homogeniser, and remaining particulate matter was removed by low-speed centrifugation, 1000×g for 5 min. Finally, the membranes were collected by high-speed centrifugation (20 000×g), suspended in binding buffer, and frozen in aliquots at −70° C.

Radioligand Binding

Frozen cell membranes were thawed, immediately rehomogenized with a Polytron homogenizer, and coupled to SPA wheat germ agglutinin beads (Amersham Life Sciences, Cardiff, England) for 30 min under continuous shaking of the tubes. After coupling, the beads were centrifuged for 10 minutes at 1000 g, and subsequently suspended in 20 ml of binding buffer per 96-well plate The binding reaction was then initiated by adding radioligand and test compounds to the bead-membrane suspension. Following incubation at room temperature, the assay plates were subjected to scintillation counting. The original SPA method was followed except for that membranes were prepared from HEK293 cells expressing the human 5-HT6 receptor instead of from HeLa cells (Dinh D M, Zaworski P G, Gill G S, Schlachter S K, Lawson C F, Smith M W. Validation of human 5-HT₆ receptors expressed in HeLa cell membranes: saturation binding studies, pharmacological profiles of standard CNS agents and SPA development. (The Upjohn Company Technical Report 7295-95-064; Dec. 27, 1995). The specific binding of [³H]LSD was saturable, while the non-specific binding increased linearly with the concentration of added radioligand (FIG. 1). [³H] LSD bound with high affinity to 5-HT₆ receptors. The K_{d} value was estimated to 2.6±0.2 nM based on four separate experiments. The total binding at 3 nM of [³H] LSD, the radioligand concentration used in the competition experiments, was typically 6000 dpm, and the specific binding more than 70%. 5-HT caused a concentration dependent inhibition of [³H] LSD binding with an over all average Ki value of 236 nM when tested against two different membrane preparations. The inter assay variability over three experiments showed a CV of 10% with an average Kᵢ values of 173 nM (SD 30) and a Hill coefficient of 0.94 (SD 0.09). The intra assay variation was 3% (n=4). All unlabelled ligands displaced the specific binding of [³H] LSD in a concentration-dependent manner, albeit at different potencies. The rank order of potency for the compounds was methiothepin (2 nM)>mianserin (190 nM)≈5-HT (236 nM)>methysergide (482 nM)>mesulergide (1970 nM).

Protein Determination

Protein concentrations were determined with BioRad Protein Assay (Bradford, M. M. A rapid and sensitive method for the quantitation of microgram quantities of protein utilizing the principle of protein-dye binding. Anal. Biochem. 1976;72:248-54). Bovine serum albumin was used as standard.

Scintillation Counting

The radioactivity was determined in a Packard TopCount™ scintillation counter (Packard Instruments, Meriden, Conn., USA) at a counting efficiency of approximately 20%. The counting efficiency was determined in separate sets of experiments.

Saturation Experiments

At least 6 concentrations in duplicates of radioligand (0.1-20 nM of [³H] LSD) were used in saturation experiments. The specific binding was calculated as the difference between total binding and non-specific binding, which was determined as the binding of radioligand in the presence of 5 μM lisuride. Bₘₐₓ and the dissociation constant, K_{d}, were determined from the non-linear regression analysis using equation 1. Lᵤ is the unbound concentration of radioligand, and is y is the amount bound.

$$y = \frac{{B}_{max} \cdot Lu}{Lu + Kd} \qquad ( equation 1 )$$

Competition Experiments

Total- and non-specific binding of radioligand was defined in eight replicates of each. Samples containing test compound were run in duplicate at 11 concentrations. Incubations were carried out at room temperature for 3 hours. The IC₅₀ value, i.e. the concentration of test compound that inhibited 50% of the specific binding of radioligand, was determined with nonlinear regression analysis and the Kᵢ value was calculated using equation 2 (Cheng Y. C. Biochem. Pharmacol. 22, 3099-3108, (1973).

$$Ki = \frac{{IC}_{50}}{1 + \frac{L}{{K}_{d}}} \\ L = concentration of radioligand \\ {K}_{d} = Affinity of radioligand \qquad ( equation 2 )$$

(b) 5-HT₆ Receptor Intrinsic Activity Assay

Antagonists to the human 5-HT₆ receptor were characterized by measuring inhibition of 5-HT induced increase in cAMP in HEK 293 cells expressing the human 5-HT₆ receptor (see Boess et al. (1997) Neuropharmacology 36: 713-720). Briefly, HEK293/5-HT₆ cells were seeded in polylysine coated 96-well plates at a density of 25,000/well and grown in DMEM (Dulbecco's Modified Eagle Medium) (without phenol-red) containing 5% dialyzed Foetal Bovine Serum for 48 h at 37° C. in a 5% CO₂ incubator. The medium was then aspirated and replaced by 0.1 ml assay medium (Hanks Balance Salt Solution containing 20 mM HEPES, 1.5 mM isobutylmethylxanthine and 1 mg/ml bovine serum albumin). After addition of test substances, 50 μl dissolved in assay medium, the cells were incubated for 10 min at 37° C. in a 5% CO₂ incubator. The medium was again aspirated and the cAMP content was determined using a radioactive cAMP kit (Amersham Pharmacia Biotech, BIOTRAK RPA559). The potency of antagonists was quantified by determining the concentration that caused 50% inhibition of 5-HT (at [5-HT]=8 times EC₅₀) evoked increase in cAMP, using the formula IC_{50,corr}=IC₅₀/(1+[5HT]/EC₅₀).

The compounds in accordance with the invention have a selective affinity to human 5-HT₆ receptors with Kᵢ and IC_{50,corr} values between 0.5 nM and 5 μM and are antagonists, agonists or partial agonists at the human 5-HT₆ receptor. The compounds show good selectivity over the human 5-HT₁ₐ, 5-HT₂ₐ, 5-HT_{2b} and 5-HT_{2c} receptors.

TABLE 2
Binding affinity (Kᵢ) at the human 5-HT₆ receptor

| Example | Kᵢ (nM) |
|---|---|
| 1 | 0.6 |
| 2 | 2.3 |
| 3 | 2.0 |

TABLE 3
Antagonist potency at the human 5-HT₆ receptor

| Example | IC_{50,corr} (nM) |
|---|---|
| 2 | 24 |
| 5 | 49 |
| 7 | 73 |
| 9 | 49 |
| 16 | 199 |
| 18 | 487 |

(c) In vivo Assay of Reduction of Food Intake

For a review on serotonin and food intake, see Blundell, J. E. and Halford, J. C. G. (1998) Serotonin and Appetite Regulation. Implications for the Pharmacological Treatment of Obesity. CNS Drugs 9:473-495.

Obese (ob/ob) mouse is selected as the primary animal model for screening as this mutant mouse consumes high amounts of food resulting in a high signal to noise ratio. To further substantiate and compare efficacy data, the effect of the compounds on food consumption is also studied in wild type (C57BL/6J) mice. The amount of food consumed during 15 hours of infusion of compounds is recorded.

Male mice (obese C57BL/6JBom-Lep^{ob} and lean wild-type C57B1/6JBom; Bomholtsgaard, Denmark) 8-9 weeks with an average body weight of 50 g (obese) and 25 g (lean) are used in all the studies. The animals are housed singly in cages at 23±1° C., 40-60% humidity and have free access to water and standard laboratory chow. The 12/12-h light/dark cycle is set to lights off at 5 p.m. The animals are conditioned for at least one week before start of study.

The test compounds are dissolved in solvents suitable for each specific compound such as cyclodextrin, cyclodextrin/methane sulfonic acid, polyethylene glycol/methane sulfonic acid, saline. Fresh solutions are made for each study. Doses of 30, 50 and 100 mg kg⁻¹ day⁻¹ are used. The purity of the test compounds is of analytical grade.

The animals are weighed at the start of the study and randomized based on body weight. Alzet osmotic minipumps (Model 2001D; infusion rate 8 μl/h) are used and loaded essentially as recommended by the Alzet technical information manual (Alza Scientific Products, 1997; Theeuwes, F. and Yam, S. I. Ann. Biomed. Eng. 4(4). 343-353, 1976). Continuous subcutaneous infusion with 24 hours duration is used. The minipumps are either filled with different concentrations of test compounds dissolved in vehicle or with only vehicle solution and maintained in vehicle pre-warmed to 37° C. (approx. 1 h). The minipumps are implanted subcutaneously in the neckiback region under short acting anesthesia (metofane/enflurane). This surgical procedure lasts approximately 5 min. It takes about 3 h to reach steady state delivery of the compound.

The weight of the food pellets are measured at 5 p.m. and at 8 p. m. for two days before (baseline) and one day after the implantation of the osmotic minipumps. The weigh-in is performed with a computer assisted Mettler Toledo PR 5002 balance. Occasional spillage is corrected for. At the end of the study the animals are killed by neck dislocation and trunk blood sampled for later analysis of plasma drug concentrations.

The plasma sample proteins are precipitated with methanol, centrifuged and the supernatant is transferred to HPLC vials and injected into the liquid chromatography/mass spectrometric system. The mass spectrometer is set for electrospray positive ion mode and Multiple Reaction Monitoring. A linear regression analysis of the standards forced through the origin is used to calculate the concentrations of the unknown samples.

Food consumption for 15 hours is measured for the three consecutive days and the percentage of basal level values is derived for each animal from the day before and after treatment. The values are expressed as mean ±SD and ±SEM from eight animals per dose group. Statistical evaluation is performed by Kruskal-Wallis one-way ANOVA using the percent basal values. If statistical significance is reached at the level of p<0.05, Mann-Whitney U-test for statistical comparison between control and treatment groups is performed.

The compounds according to the invention show an effect in the range of 50-200 mg/kg.

1. A compound of Formula (I) 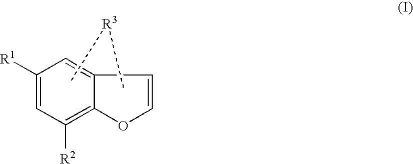
wherein
one of R¹ and R² is selected from Formula (II) or (III) 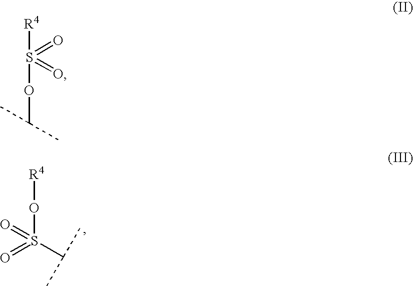
while the other one of R¹ and R² is selected from group of Formula (IV)-(XV) 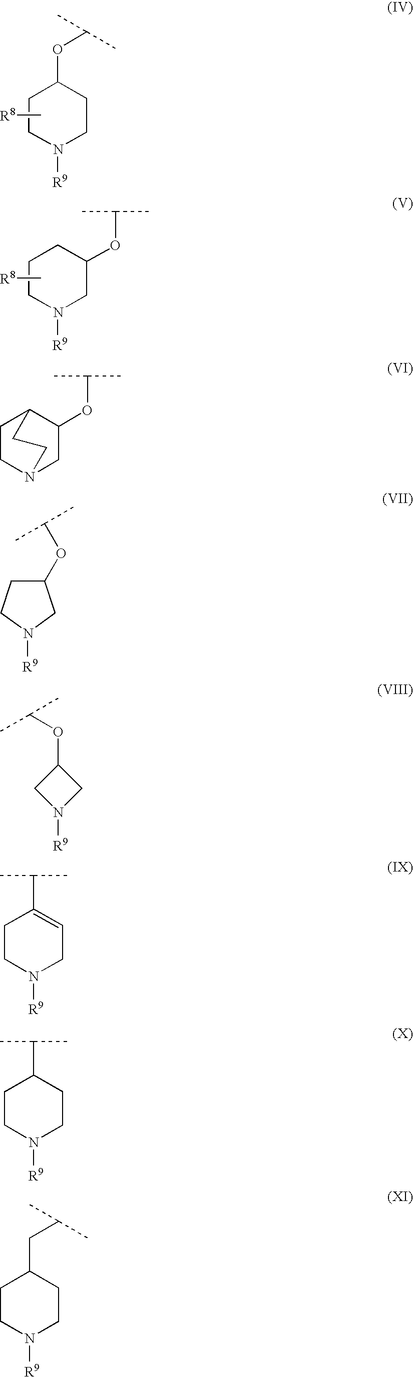 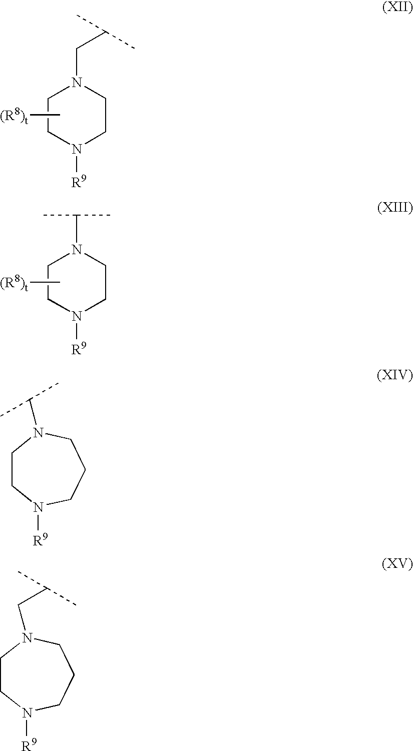
wherein
t is 0, 1, or 2;
R⁸ is each independently
(a) hydrogen,
(b) methyl, or
(c) ethyl, and
when t=2, the R⁸ groups can be attached to the same or different carbon atom(s);
R⁹ is
(a) H,
(b) C₁₋₆ alkyl, or
(c) benzyl;
R³ is selected from
(a) hydrogen,
(b) C₁₋₄-alkyl,
(c) halogen, and
(d) C₁₋₄-alkoxy,
wherein the said R³ group is attached to a carbon atom in the 5-membered or the 6-membered ring;
R⁴ is selected from
(a) aryl,
(b) heteroaryl,
(c) heterocyclyl, provided that R¹ or R² is selected from a group of Formula (II),
(d) aryl-C₁₋₂-alkyl, provided that R¹ or R² is selected from a group of Formula (II), and
(e) cinnamyl, provided that R¹ or R² is selected from the group of Formula (II),
wherein any aryl and heteroaryl is optionally substituted in one or more positions with a substituent selected from:
(a) halogen,
(b) C₁₋₆-alkyl,
(c) CF₃,
(d) C₁₋₆-alkoxy,
(e) C₂₋₆-alkenyl,
(f) phenyl,
(g) phenoxy,
(h) benzyloxy,
(i) benzoyl,
(j) —OCF₃,
(k —CN,
(l) hydroxy-C₁₋₄-alkyl,
(m) —CH₂—(CH₂)ₚF, wherein p is 0, 1, 2, or 3,
(n) —CHF₂,
(o) —NR⁵R⁵,
(p) —NO₂,
(q) —CONR⁵R⁵,
(r) —NHSO₂R⁷,
(s) —NR⁶COR⁷,
(t) —SO₂NR⁶R⁷,
(u) —C(═O)R⁷,
(v) —CO₂R⁶,
(z) —S(O)ₙR⁷, wherein n is 1 or 2,
(aa) C₁₋₆-alkylthio,
(ab) —SCF₃,
(ac) C₂₋₄-alkynyl, and
(ad) hydroxyl;
R⁵ is each independently selected from
(a) H,
(b) C₁₋₆-alkyl, and
(c) C₃₋₇-cycloalkyl,
wherein the two R⁵ groups together with the nitrogen to which they are attached form a heterocyclic ring; and when the two R⁵ groups form a piperazine ring, the hydrogen bearing nitrogen of the piperazine ring may be optionally substituted with a group selected from
(a) C₁₋₄-alkyl,
(b) 2-cyanoethyl,
(c) hydroxy-C₂₋₄-alkyl,
(d) C₃₋₄-alkenyl,
(e) C₃₋₇-cycloalkyl,
(f) C₃₋₇-cycloalkyl-C₁₋₄-alkyl, and
(g) C₁₋₄-alkoxy-C₂₋₄-alkyl;
R⁶ is each independently selected from
(a) hydrogen, and
(b) C₁₋₄-alkyl; and
R⁷ is independently selected from
(a) C₁₋₆-alkyl
(b) aryl, and
(c) heteroaryl,
wherein any heteroaryl or aryl residue is optionally substituted with a substituent selected from
(a) halogen,
(b) C₁₋₄-alkyl,
(c) C₁₋₄-alkylthio,
(d) C₁₋₄-alkoxy,
(e) —CF₃, and
(f) —CN;
and a pharmaceutical acceptable salt thereof.

2. A compound according to claim 1, wherein R¹ is of Formula (III)

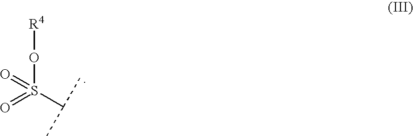

3. A compound according to claim 1 or 2, wherein R⁹ is hydrogen or methyl.

4. A compound according to claim 1 or 2, wherein R² is selected from piperazinyl; homopiperazinyl; 2,6-dimethylpiperazinyl; 3,5-dimethylpiperazinyl; 2,5-dimethylpiperazinyl; 2-methylpiperazinyl; 3-methylpiperazinyl; 2,2-dimethylpiperazinyl; 3,3-dimethylpiperazinyl; piperidinyl; 1,2-unsaturated piperidinyl; 4-pyrrolidin-3-yloxy, 4-piperidinyloxy, and piperazinylmethyl.

5. A compound according to claim 1 or claim 2, wherein R² is piperazinyl.

6. A compound according to claim 1 or claim 2, wherein R³ is hydrogen.

7. A compound according to claim 1 or claim 2, wherein R⁴ is phenyl,
wherein the phenyl is optionally substituted in one or more positions with a substituent selected from;
(a) halogen,
(b) C₁₋₆-alkyl,
(c) CF₃, and
(d) C₁₋₆-alkoxy.

8. A compound according to claim 1 selected from: 2-Methoxy-5-methylphenyl 7-piperazin-1-yl-1-benzofuran-5-sulfonate, 2-Chlorophenyl-7-piperazin-1-yl-1-benzofur 5-sulfonate, 2-(Trifluoromethyl)-phenyl 7-piperazin-1-yl-1-benzofuran-5-sulfonate, Pyridin-3-yl 7-piperazin-1-yl-1benzofuran-5-sulfonate, 2-Methoxy-5-methylphenyl 7-[(4-methylpiperazin-1-yl)methyl]-1-benzofuran-5-sulfonate, 2-Methoxy-5-methylphenyl 7-{[(3R)-3-methylpiperazin-1-yl]methyl}-1-benzofuran-5-sulfonate, Pyridin-3-yl 7-(4-methylpiperazin-1-yl)-1-benzofuran-5-sulfonate, 2,3-Dimethoxyphenyl 7-(4-methylpiperazin-1yl)-1-benzofuran-5-sulfonate, 2,3-Dimethoxyphenyl 7-[(3R)-3-methylpiperazin-1-yl]-1-benzofuran-5-sulfonate, 2,3-Dimethoxyphenyl 7-[(3S)-3-methylpiperazin-1-yl]-1-benzofuran-5-sulfonate, 3,5-Dimethoxyphenyl 7-(4-methylpiperazin-1-yl]-1-benzofuran-5-sulfonate, 3,5-Dimethoxyphenyl 7-[(3R)-3-methylpiperazin-1-yl]-1-benzofuran-5-sulfonate, 3,5-Dimethoxyphenyl 7-[(3S)-3-methylpiperazin-1-yl]-1benzorfuran-5-sulfonate, 2-Methoxy-5-methylphenyl 7-{[(3 S)-3-methylpiperazin-1-yl]methyl}-1benzofuran-5-sulfonate, 2-(Aminocarbonyl)phenyl 7-{[(3S)-3-{[(3 S)-3-methylpiperazin-1-yl]methyl}-1benzofuran-5-sulfonate, 2-(Aminocarbonyl)phenyl-7-{[(3R)-3-methylpiperazin-1-yl]methyl}-1benzofuran-5-sulfonate, 2-Methoxy-5-methylphenyl 7-(piperazin-1-ylmethyl)-1-benzofuran-5-sulfonate, 2-methoxy-5-methylphenyl 7-(1,4-diazepan-1-ylmethyl)-1-benzofuran-5-sulfonate, and the pharmaceutically acceptable salts thereof.

9. A compound according to claim 1 wherein:
R¹ has Formula (III) 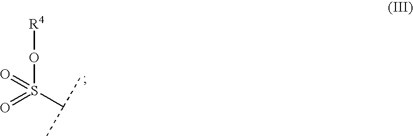
R² is selected from piperazinyl, homopiperazinyl, 3-methylpiperazinyl, 4-methylpiperazin-1-yl, homopiperazin-1ylmethyl, 3-methylpiperazin-1-ylmethyl, and piperazin-1ylmethyl;
R³ is hydrogen; and
R⁴ is selected from pyridinyl and phenyl,
wherein phenyl is optionally independently substituted in one or more positions with a substituent selected from:
(a) halogen selected from fluorine and chlorine
(b) C₁₋₄-alkyl,
(c) CF₃,
(d) C₁₋₄-alkoxy, and
(q) CONR⁵R⁵.

10. A compound of claim 1 wherein R¹ has Formula (III) 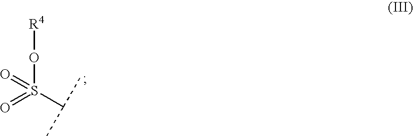
R² is selected from piperazinyl, homopiperazinyl, 3-methylpiperazinyl, 4-methylpiperazin-1-yl, homopiperazin-1ylmethyl, 3-methylpiperazin-1-ylmethyl, and piperazin-1ylmethyl;
R³ is hydrogen; and
R⁴ is selected from pyridinyl and phenyl,
wherein phenyl is optionally independently substituted in one or more positions with a substituent selected from:
(a) chlorine
(b) methyl,
(c) CF₃,
(d) methoxy, and
(q) CONH₂.

11. A pharmaceutical formulation containing a compound according claim as an active ingredient, in combination with a pharmaceutically acceptable diluent or carrier.

12. A method for the treatment or prophylaxis of obesity, type II diabetes, and/or disorders of the central nervous system, which comprises administering to claim 1.

13. A method of claim 12 wherein the central nervous system disorder is selected from: anxiety, depression, panic attacks, memory disorders, cognitive disorders, epilepsy, sleep disorders, migraine, anorexia, bulimia, binge eating disorders, obsessive compulsive disorders, psychoses, Alzheimer's disease, Parkinson's disease, Huntington's chorea, schizophrenia, attention deficit hyperactive disorder, and withdrawal from drug abuse.

14. A method for reducing body-weight or reducing body weight gain, the method comprising administering to a subject in need thereof an effective amount of a compound according to claim 1.

15. A method for modulating 5-HT₆ receptor activity, comprising administering to a subject in need thereof an effective amount of a compound according to claim 1.

16. A method comprising combining a compound of claim 1 with a pharmaceutically acceptable diluent or carrier.

17. A process for the synthesis of a compound of claim 1, comprising:
(a) preparing a 7-substituted-2,3-dihydrobenzofuran-5-sulfonyl chloride from 2,3-dihydrobenzofuran-5-sulfonyl chloride and iodine monochloride;
(b) oxidating the 7-substituted-2,3-dihydrobenzofuran-5-sulfonyl chloride with N-bromosuccinimide to provide 7-substituted benzofuran-5-sulfonyl chloride;
(c) reacting the 7-substituted benzofuran-5-sulphonyl chloride intermediate, selected from: 7-iodo-benzofuran-5-sulphonyl chloride, 7-bromo-benzofuran-5-sulphonyl chloride, 7-formyl-benzofuran-5-sulphonyl chloride or 7-hydroxy-benzofuran-5-sulphonyl chloride, with a hydroxy compound corresponding to R⁴OH, and
(d) reacting the product from step c) with corresponding group selected from Formula (IV)-(XV); and optionally thereafter forming a pharmaceutically acceptable salt of the compound of Formula (I).

18. A process for the synthesis of a compound according claim 1, wherein R¹ is selected from Formula (III) and R² is selected from Formula (XIII) and (XIV), the process comprising:
(a) reacting a 7-halo substituted benzoftiran derivative of Formula (IIa), 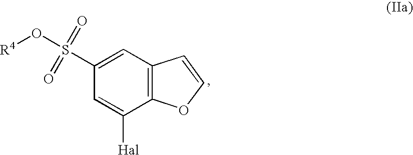
Hal is selected from chloro, bromo and iodo, with an appropriate secondary amine, or a protected derivative thereof, in the presence of a palladium catalyst together with an auxilliary ligand and a base, to give, optionally after deprotection, a compound of Formula (I), wherein R² is selected from Formula (XIII) and (XIV); and optionally thereafter forming a pharmaceutically acceptable salt of the compound of Formula (I).

19. A process for the synthesis of a compound according claim 1, wherein RI is selected from Formula (III) and R² is selected from Formula (XII) and (XV), the process comprising:
(a) reacting a 7-halo substituted benzofuran derivative of Formula (IIa), 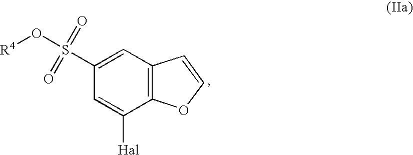
and Hal is selected from chloro, bromo and iodo, with a metal cyanide salt, to give a compound of Formula (IIIa) 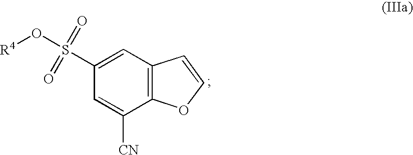
(b) reacting the compound of Formula (IIIa) with a reducing agent, to give a compound of Formula (IVa) 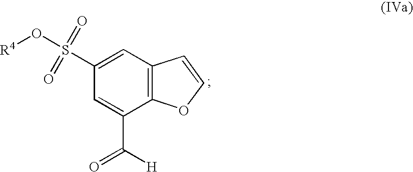
(c) reacting the compound of Formula (IVa) with an appropriate secondary amine, or a protected derivative thereof, in the presence of a suitable reducing agent such as NaBH₄, NaBH₃CN or sodium triacetoxyborohydride [NaB(OAc)₃)H], to give, optionally after deprotection, a compound of Formula (I) wherein R² is selected from formula (XII) and (XV); and optionally thereafter forming a pharmaceutically acceptable salt of the compound of formula (I).

20. A process for the synthesis of a compound according claim 1, wherein R¹ is selected from Formula (III) and R² is selected from formula (XII) and (XV), the process comprising:
(a) reacting a 7-halo substituted benzofuran derivative of Formula (IIa), 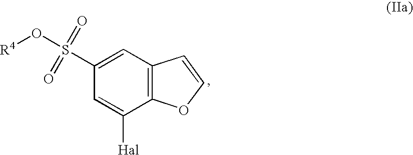
Hal is selected from chloro, bromo and iodo,
preferably iodo, with tributyl(vinyl)stannane in the presence of a palladium complex such as bis(triphenylphosphine)palladium(II) diacetate [Pd(PPh₃)₂OAc₂] as a catalyst, to give a compound of formula (Va) 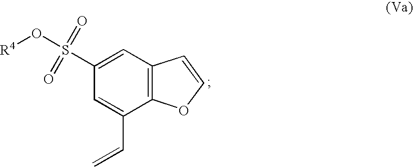
(b) reacting the compound of formula (Va) with osmium tetroxide (OsO₄) and sodium periodate, to produce the aldehyde derivative of formula (IVa) 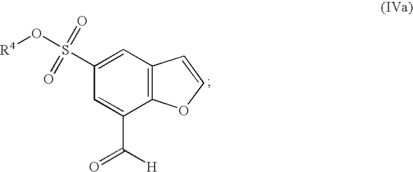
(c) reacting a compound of formula (IVa) with an appropriate secondary amine, or a protected derivative thereof, in the presence of a suitable reducing agent such as NaBH₄, NaBH₃CN or sodium triacetoxyborohydride [NaB(OAc)₃)H], to give, optionally after deprotection, a compound of Formula (I) wherein R² is selected from formula (XII) and (XV); and optionally thereafter forming a pharmaceutically acceptable salt of the compound of formula (I).
